(19) United States
(12) Patent Application Publication
(10) Pub. No.: US 2009/0169576 A1
(43) Pub. Date: Jul. 2, 2009

(54) INFLUENZA COMBINATORIAL ANTIGEN VACCINE

(76) Inventors: Roberto Crea, San Mateo, CA (US); Mario H. Genero, Buenos Aires (AR); Guido Cappuccilli, San Mateo, CA (US); Randy Shen, Sunnyvale, CA (US)

Correspondence Address: King & Spalding LLP, P.O. Box 889, Belmont, CA, 94002-0889, US

(21) Appl. No.: 12/091,423

(22) PCT Filed: Oct. 26, 2006
(86) PCT No.: PCT/US2006/042411

§ 371 Date: Aug. 27, 2008

Related U.S. Application Data
(60) Provisional application No. 60/730,658, filed on Oct. 26, 2005.

(51) Int. Cl. A61K 39/145; A61P 31/16; A61P 37/04

(52) U.S. Cl. 424/186.1

(57) ABSTRACT

The invention provides a combinatorial influenza vaccine composition for use in providing prophylactic protection in humans against influenza viruses or animals, and a method of producing the vaccine.

### FIELD OF THE INVENTION

The present invention relates to a polypeptide composition for use in vaccinating humans against Influenza.

### BACKGROUND OF THE INVENTION

Host defense is a hallmark of vertebrate immune systems. To this end, antibodies perform numerous functions in the defense against pathogens. For instance, antibodies can neutralize a biologically active molecule, induce the complement pathway, stimulate phagocytosis (opsonization), or participate in antibody-dependent cell-mediated cytotoxicity (ADCC).

If the antibody binds to a site critical for the biological function of a molecule, the activity of the molecule can be neutralized. In this way, specific antibodies can block the binding of a virus or a protozoan to the surface of a cell. Similarly, bacterial and other types of toxins can be bound and neutralized by appropriate antibodies. Moreover, regardless of whether a bound antibody neutralizes its target, the resulting antigen-antibody complex can interact with other defense mechanisms, resulting in destruction and/or clearance of the antigen.

Vaccines are designed to stimulate the immune system to protect against microorganisms such as viruses. When a foreign substance invades the body, the immune system activates certain cells to destroy the invader. This activation of the immune system involves two main types of cells: B cells and T cells. In humoral defense, B cells make antibodies, molecules that attach to and neutralize viruses floating free in the bloodstream, thereby preventing the viruses from infecting other cells. In cell mediated defense, T cells can be helper cells or killer cells. Helper T cells organize the immune response. Killer T cells, known as CD8+ CTLs, attack cells infected by viruses.

Many viruses are capable of great antigenic variation, and large numbers of serologically distinct strains of these viruses have been identified. As a result, a particular strain of a virus becomes insusceptible to immunity generated in the population by previous infection or vaccination. During the past century, influenza viruses with hemagglutinin (HA) glycoproteins from 3 of the 15 influenza A virus subtypes (H1-H15) emerged from avian or animal hosts to cause worldwide epidemics: in 1918, H1; in 1957, H2 and in 1968, H3 (WHO Memorandum (1980) Bull. W. H. O. 58, 585-591). Attempts to control influenza by vaccination has so far been of limited success and are hindered by continual changes in the major surface antigen of influenza viruses, the hemagglutinin (HA) and neuraminidase (NA), against which neutralizing antibodies are primarily directed (Caton et al. (1982) Cell 31:417; Cox et al. (1983) Bulletin W.H.O. 61:143; Eckert, E. A. (1972) J. Virology 11:183). The influenza viruses have the ability to undergo a high degree of antigenic variation within a short period of time. It is this property of the virus that has made it difficult to control the seasonal outbreaks of influenza throughout the human and animal populations. Receptor binding, the initial event in virus infection, is a major determinant of virus transmissibility that for influenza viruses is mediated by the hemagglutinin membrane glycoprotein (HA) (Ya Ha et al., X-ray structures of H5 avian and H9 swine influenza virus hemagglutinins bound to avian and human receptor analogs. PNAS 98: 11181-11186).

Through seralogic and sequencing studies, two types of antigenic variations have been demonstrated in influenza A viruses. Antigenic shift occurs primarily when either HA or NA, or both, are replaced in a new viral strain with a new antigenically novel HA or NA. The occurrence of new subtypes created by antigenic shift usually results in pandemics of infection.

Antigenic drift occurs in influenza viruses of a given subtype. Amino acid and nucleotide sequence analysis suggests that antigenic drift occurs through a series of sequential mutations, resulting in amino acid changes in the polypeptide and differences in the antigenicity of the virus. The accumulation of several mutations via antigenic drift eventually results in a subtype able to evade the immune response of a wide number of subjects previously exposed to a similar subtype. In fact, similar new variants have been selected experimentally by passage of viruses in the presence of small amounts of antibodies in mice or chick embryos. Antigenic drift gives rise to less serious outbreak, or epidemics, of infection. Antigenic drift has also been observed in influenza B viruses.

Influenza viruses are composed of eight segments of single stranded RNA of negative polarity, totaling approximately 14 kilobases, encode for at least 10 viral proteins. The three viral polymerases (PB1, PB2 and PA) are encoded by approximately half of the total genome by RNA segments 1, 2 and 3 respectively. RNA segment 5 encodes the NP protein. The three-polymerase subunits, the NP and the vRNA then associate as virions in infected cells in the form of viral ribonucleoprotein particles (vRNPs). RNA segments 4 and 6 encode for the HA and NA genes, respectively. The two smallest RNA segments (7 and 8) encode two genes each with overlapping reading frames, which are generated by splicing of the co-linear mRNA molecules. In addition to M1, RNA segment 7 encodes for the M2 protein, which has ion channel activity and is embedded in the viral envelope. Segment 8 encodes for NS1, a nonstructural protein that blocks the host's antiviral response, and NS2 or NEP that participates in the assembly of virus particles.

The Influenza viruses are enclosed in a lipid envelope that is acquired in the final step of virus assembly. The viruses bud from the host cell membranes where the virally encoded glycoproteins, HA and NA, have accumulated. After budding, the Influenza envelope is spiked with HA and is the most abundant protein on the virus surface. In subsequent infection of new host cells, HA plays an important role in virus recognition, attachment and membrane fusion.

After host cell receptor attachment, the virus is then internalized by endocytosis. Acidification of the endosome then leads to conformational changes of HA protein fusing the viral and the endosomal membranes. Endosomal acidification also activates the ion channel activity of influenza matrix protein 2 (M2) whereupon an inward current of protons into the virion's interior that triggers the disassembly of matrix protein 1 (M1) from the vRNPs.

The released vRNPs, composed of viral RNA (vRNA) and nucleocapsid proteins (NP), are then transported to the nucleus for virus transcription and replication. Two different populations of positive sense RNAs are synthesized from vRNA templates, messenger RNAs (mRNAs) and complementary RNAs (cRNAs). The first step is the synthesis and transcription of cRNA representing full-length copies of vRNAs. The virus carries its' own RNA replicase complex (PB1, PB2 and PBa) as the host cell lacks protein(s) capable of performing this function. Viral mRNAs are then primed by 5′ capping fragments and polyadenylated for export and proper protein translation in the cytoplasm.

The second step in viral replication is the synthesis of progeny vRNA genomes from cRNAs templates. As the infection cycle progresses, when sufficient M1 matrix protein and nucleocapsid (NP) have been produced, the newly synthesized vRNPs are then exported out of the nucleus and assembled into full virus particles. The final assembly steps occur at the plasma membrane incorporating the newly synthesized HA, NA, and M2 proteins. HA and NA are present as homotrimers and homotetramers, respectively on the viral envelope. Within the envelope, M1 and NP proteins protect the vRNA.

The mature HA homotrimer is initially processed from a single polypeptide precursor, HA0. HA0 is subsequently cleaved into the subunits, HA1 and HA2.

Both HA1 and HA2 subunits are glycosylated (see below) and are linked by a single disulphide bond between them. Each HA monomer consists of a globular head region connected to a fibrous stem domain. At the N-terminal end of the HA2 chain is the fusion peptide which is critical for subsequent membrane fusion events that lead to infection. Both regions carry N-linked oligosaccharide side chains, with those attached to the stalk region being highly conserved while those at the tip of the molecule showing considerable variation.

Sialic acid is a key component as infection is initiated by multivalent binding of HA on the viral envelope to the sialic acid-terminated oligosaccharides displayed on the host cell surface. Influenza host cell specificity is attributable, in part, to the virus being able to distinguish between Sia-alpha-2,3-Gal and Sia-alpha-2,6-Gal linkages and also between the N-acetylneuramic acid (Neu5Ac) and N-glycolylneuramic acid (Neu5Gc) forms of sialic acid. One of the differences between avian and human influenza strains is that human influenza targets Sia-2,6-linked NeuAc whereas the avian preferred ligand is Sia-alpha-2,3-Gal (G. N. Rogers, J. C. Paulson, Virology 127, 361 (1983). Thus, HA binding preference for linkage types correlates with the species specificity. The 15 avian antigenic HAs subtypes bind preferentially to this α-2,3-linkage form which is sialosaccharide that predominates in avian enteric tracts. The human H1, H2, and H3 subtypes responsible for the 1918, 1957, and 1968 pandemics respectively, recognize α-2,6-linked sialic acid, the major form found on cells of the human respiratory tract. Interestingly, serving as a cross-species reservoir pool, swine influenza viruses are reported to bind both sialic acid in α-2,6- and sometimes also in α-2,3-linkage and sialic acid and both linkage have is detected in porcine trachea cells. (L. G. Baum, J. C. Paulson, Acta Histochem Suppl 40, 35 (1990). J. N. Couceiro, J. C. Paulson, L. G. Baum, Virus Res 29 155 (1993).

After binding and cellular endocytosis, NA then acts to cleave terminal sialic acids to facilitate virus release from endocytotic vesicles. Functional NA protein is configured as a homotetramer. The neuraminidase protein has a box-shaped globular head with four catalytic sites that allow the cleavage of sialic acid linkages. The active site of the NA enzyme is usually localized in 15 invariant amino on the terminal knob (Colman, P. M., Varghese, J. N. & Laver, W. G. Structure of the catalytic and antigenic sites influenza virus neuraminidase. Nature 303, 41-44 (1983)). Amino acid positions important for antigenic drift have been identified for the N2 subtype as well as other regions likely to be involved in virus-host interactions. Sequence data according to the study of Elodie Ghedin et al., indicate that once residue 197—an antigenic site, mutated from 197H to 197D early in the 1999-2000 influenza season, it was accompanied by the mutation R249K. This residue is probably not in a functional site but may be functionally compensating by maintaining the accessibility of surface residues. Residue 199 interestingly switched (E199K) for the 2003-2004 influenza season for the majority of the isolates, except for the two isolates corresponding to the minor non-reassorted clade (A/New York/269/2003 and A/New York/32/2003) (Elodie Ghedin et al., Large-scale sequencing of human influenza reveals the dynamic nature of viral genome evolution. Nature 437, 1162-1166 (20 Oct. 2005)).

Influenza oligosaccharide monomer components, branching structure and polypeptide sites for both NA and HA-glycosylation are determined in part, by the glycosylation signals and host cell glycosylation machinery. Carbohydrate additions can either occur as N-linked or O-linked glycosylations. The early events of N-glycosylation occur in the endoplasmic reticulum (ER). First, an oligosaccharide chain comprising fourteen sugar residues is constructed on a lipid carrier molecule. After initial translation, a targeting sequence translocates the nascent peptide into the ER.

The entire oligosaccharide chain (glycan) is then transferred to the amide group of the asparagine residue (Asn) in a reaction catalyzed by a membrane bound glycosyltransferase enzyme. An amino acid triplet Asn-X-Y characterizes N-glycosylation sites in eukaryotic polypeptides, wherein X is any amino acid except Pro and Y is Ser or Thr. The N-linked glycan is further processed both in the ER and in the Golgi apparatus. Generally, there is the removal of some of the sugar residues and/or addition of other sugar residues in reactions catalyzed by specific modifying glycosidases and glycosyltransferases.

Peptides may also be modified by addition of O-linked glycans, also called mucin-type glycans because of their prevalence on mucinous glycopeptide. Unlike N-glycans that are linked to asparagine residues and are formed by en bloc transfer of oligosaccharide from lipid-bound intermediates, O-glycans are linked primarily to serine and threonine residues and are formed by the stepwise addition of single sugars typically N-acetylgalactosamine in mammals. O-linked glycosylation is also initiated in the Golgi and can vary in size from a single N-acetylgalactosamine residue to oligosaccharides comparable in size to N-linked glycans.

The pattern of HA glycosylation regulates, in part, host cell receptor specificity. Studies have reported that certain glycosylation sites are conserved between various animals and humans and therefore are of functional importance. For example in the stem region, N-glycosylation sites at Asn12 and Asn478 have been found to be very conserved in many HA protein sequences. Some regions of the HA protein must be free of oligosaccharide in order for maintenance of HA whereas oligosaccharides near the cleavage site modulate proteolytic activity. Deletions and structural modifications to HA-oligosaccharides influence host cell attachment and change viral replication dynamics.

The biological functions of oligosaccharides on HA have also been implicated in viral pathogenicity by shielding the HA from host proteases and neutralize antibodies. Glycosylation, is of significant importance for this invention, as it also determines the antigenic epitopes presented by the vaccine candidates.

### SUMMARY OF THE INVENTION

The present invention includes, in one aspect, an influenza vaccine composition comprising, in a physiological carrier, a set of peptides identified by a sequence selected from the group consisting of SEQ ID NOS: 1-13, where each of SEQ ID NOS. 1-13 represents a selected region from one of the major influenza surface antigens, hemagglutinin (HA) and neuraminidase (NA), as follows:

Seq No 1 HA: (residues 211 to 240; including the 190-helix (residues 223-231); Seq No 2 HA: (residues 151 to 180; including the 130-loop (residues 165-168); Seq No 3 HA: (residues 151 to 180; including the 220-loop (residues 254-261); Seq No 4 HA: (residues 366 to 394; including the “Cleavage site” {residue 380} where for full infectivity, the single chain (HA0) is cut into two chains for full infectivity; and Seq No 5: NA (residues 18 to 437), Seq No 6: NA (residues 321 to 341), Seq No 7: NA (residues 342 to 400), Seq No 8: (residues 153 to 185), Seq No 9: (residues 209 to 232), Seq No 10: (residues 330 to 369), Seq No 11: (residues 369 to 398), Seq No 12: (residues 399 to 434), and Seq No 13: (residues 435 to 460).

The set of peptides may include all or a subset of the peptides contained within the selected SEQ ID NOS: 1-13. In one general embodiment, the vaccine includes a subset of 5-100, preferably 5-50, peptide antigens having amino acid sequences contained in the set of peptides identified by a sequences selected from the group consisting of SEQ ID NOS: 1-13, and selected from the total set of antigen peptides defined by one of SEQ ID NOS: 1-13 by the steps of:

(i) limiting the influenza-strain variants examined for amino acid variations within one of the regions defining SEQ ID NOS: 1-13 to a selected one of the human-infective influenza subtypes identified by H1, H2, H3, H5; H7, and H9,

(ii) optionally, limiting the influenza-strain variants examined for amino-acid variation to those associated with a particular geographic region; and

(iii) selecting for the subset, peptide antigens having amino acid sequences that represent existing amino acid variants examined in steps (i) and (ii), and those that represent most-likely amino acid mutations of one or more of the existing variants, such that the total number of peptide antigens selected for the subset is between 5 and 50.

One exemplary influenza vaccine composition contains a subset of peptide antigens contained in SEQ ID NO. 14, and is selected from the total set of antigen peptides defined by SEQ ID NO: 2 by a selection in which:

step (i) includes limiting the influenza-strain variants examined for amino acid variations within SEQ ID NO: 2 to H5N1 subtypes of the virus;

step (ii) includes limiting the influenza-strain variants examined for amino-acid variation to those associated with human influenza infections in Indonesia and Thailand, and

step (iii) includes selecting for the subset, 6 peptide antigens having amino acid sequences that represent existing amino acid variants examined in steps (i) and (ii), and 31 single-amino acid mutations of one or more of the existing variants, such that the total number of peptide antigens selected for the subset is 37 and the sequence of the subset is given by SEQ ID NO: 14.

Another exemplary influenza vaccine composition contains a subset of peptide antigens contained in SEQ ID NO. 14, and is selected from the total set of antigen peptides defined by SEQ ID NO: 2 by a selection in which:

step (i) includes limiting the influenza-strain variants examined for amino acid variations within SEQ ID NO: 2 to H5N1 subtypes of the virus;

step (ii) includes limiting the influenza-strain variants examined for amino-acid variation to those associated with human influenza infections in Indonesia an Thailand, and

step (iii) includes selecting for the subset, 5 peptide antigens having amino acid sequences that represent existing amino acid variants examined in steps (i) and (ii), and 11 single-amino acid mutations of one or more of the existing variants, such that the total number of peptide antigens selected for the subset is 16 and the sequence of the subset is given by SEQ ID NO: 20.

Also forming an aspect of the invention is a method of producing an influenza vaccine composition comprising, in a physiological carrier, a subset of 5-50 peptide antigens having amino acid sequences contained in the set of peptides identified by a sequences selected from the group consisting of SEQ ID NOS: 1-13, where each of SEQ ID NOS. 1-13 represents a selected region from one of the major influenza surface antigens, hemagglutinin (HA) and neuraminidase (NA). The method includes the steps of:

(i) limiting the influenza-strain variants examined for amino acid variations within one of the regions defining SEQ ID NOS: 1-13 to a selected one of the human-infective influenza subtypes identified by H1, H2, H3, and H5;

(ii) optionally, limiting the influenza-strain variants examined for amino-acid variation to those associated with a particular geographic region; and

(iii) selecting for the subset, peptide antigens having amino acid sequences that represent existing amino acid variants examined in steps (i) and (ii), and those that represent most-likely single-amino acid mutations of one or more of the existing variants, such that the total number of peptide antigens selected for the subset, i.e., where each peptide antigen is present in an amount likely to contribute to an immune response in the vaccinated subject, is between 5 and 50.

These and other and features of the present invention will become more fully apparent when the following detailed description of the invention is read in conjunction with the accompanying drawings.

### BRIEF DESCRIPTION OF THE DRAWINGS

FIG. 1a shows the HA protein sequence from amino acid positions 1 to 160 in each of 15 subtypes of influenza A, where the alignment is informed by subtypes H1, H3, H5, and H9. The first ten amino acids of SEQ No 2 HA is shown encompassing HA positions 150 to 160.

FIG. 1b. shows the HA protein sequence from amino acid positions 161 to 320 in each of 15 subtypes of influenza A. SEQ No1 HA as shown by the arrow is defined by HA amino residues 211-240. SEQ No3 HA as shown by the arrow is defined by HA amino residues 241-270. SEQ No2 HA as shown (continuing from FIG. 1a) by the arrow is defined by HA amino residues 161-180.

FIG. 1c. 1a shows the HA protein sequence from amino acid positions 321 to 480 of HA protein sequences representative of each influenza A subtype. SEQ No4 HA as shown by the arrow is defined by HA amino residues 366-394.

FIG. 1d. Alignment of amino acid residues 481 to 604 of HA protein sequences representative of each influenza A subtype.

FIG. 2a. shows an alignment of the NA protein sequence of the indicated amino acid positions 18 to 437 from thirty isolates of H3N2, SEQ No 5 NA illustrates the alignment of those NA positions.

FIG. 2b. shows an alignment of the NA protein sequence of the indicated amino acid positions 18 to 437 of H3N2 isolates 31 to 60, SEQ No 5 NA illustrates the alignment of those NA positions.

FIG. 2c. shows an alignment of the NA protein sequence of the indicated amino acid positions 18 to 437 of H3N2 isolates 61 to 90. SEQ No 5 NA illustrates the alignment of those NA positions.

FIG. 2d. shows an alignment of the NA protein sequence of the indicated amino acid positions 18 to 437 of H3N2 isolates 91 to 124. SEQ No 5 NA illustrates the alignment of those NA positions.

FIG. 3. shows the NA protein sequence from amino acid positions 321 to 400 in various influenza A isolates. SEQ No6 NA as shown by the arrow is defined by NA amino residues 321-341 and SEQ No 7 NA as shown by the arrow is defined by NA amino residues 342-400.

FIG. 4. shows the NA protein sequence from amino acid positions 153 to 185 in 10 subtypes of influenza A. SEQ No 8 HA illustrates the alignment of those NA positions encompassing NA positions 153 to 185. Also shown is the position of Neuraminidase Antigenic site 1 (residue 170).

FIG. 5. shows the NA protein sequence from amino acid positions 153 to 185 in 10 subtypes of influenza A. SEQ No 8 HA illustrates the alignment of those NA positions encompassing NA positions 153 to 185. Also shown is the position of Neuraminidase Antigenic site 1 (residue 170).

FIG. 6. shows an alignment of the indicated influenza isolates for NA protein amino acid residues 330 to 369 including neuraminidase antigenic sites, 3, 4, 5 and 6 (indicated by the arrows at residues 346, 353, 357-358, and 361-367 respectively). SEQ No 10 NA as shown by the arrow is defined by NA amino residues 330 to 369.

FIG. 7. shows an alignment of the indicated influenza isolates for NA protein amino acid residues 369 to 460 including neuraminidase antigenic sites, 7, 8, and 9 (indicated by the arrows at residues 387-389, 420-421, and 454-457 respectively). SEQ No 11 NA as shown by the arrow is defined by NA amino residues 369 to 398, SEQ No 12 NA as shown by the arrow is defined by NA amino residues 399 to 434, SEQ No 13 NA as shown by the arrow is defined by NA amino residues 435 to 460.

### DETAILED DESCRIPTION OF THE INVENTION

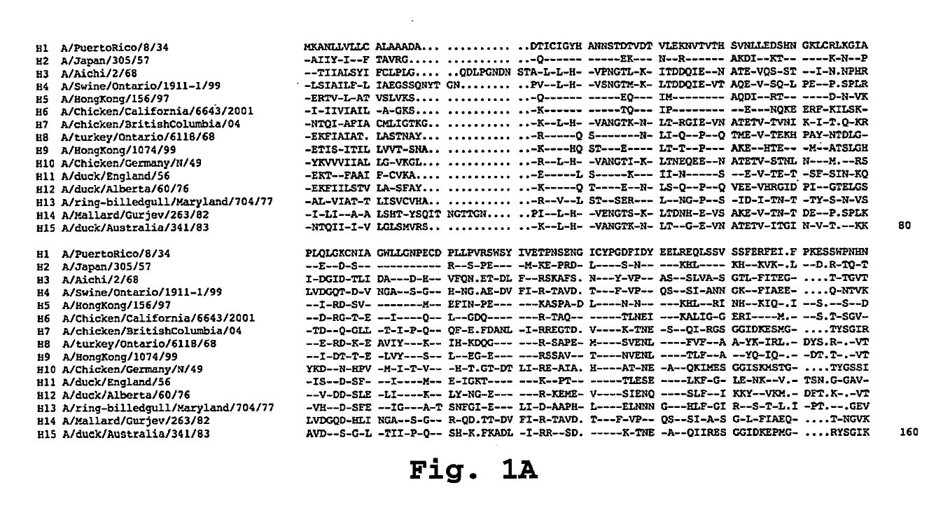

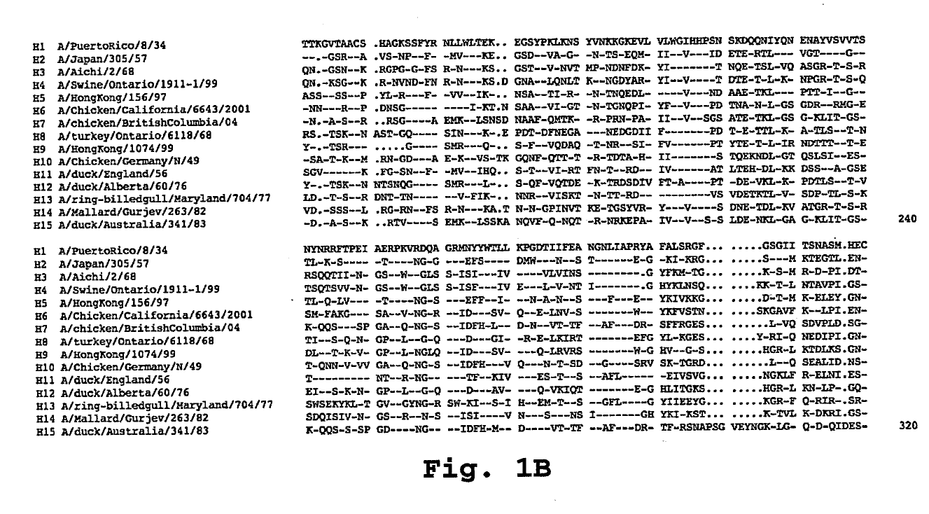

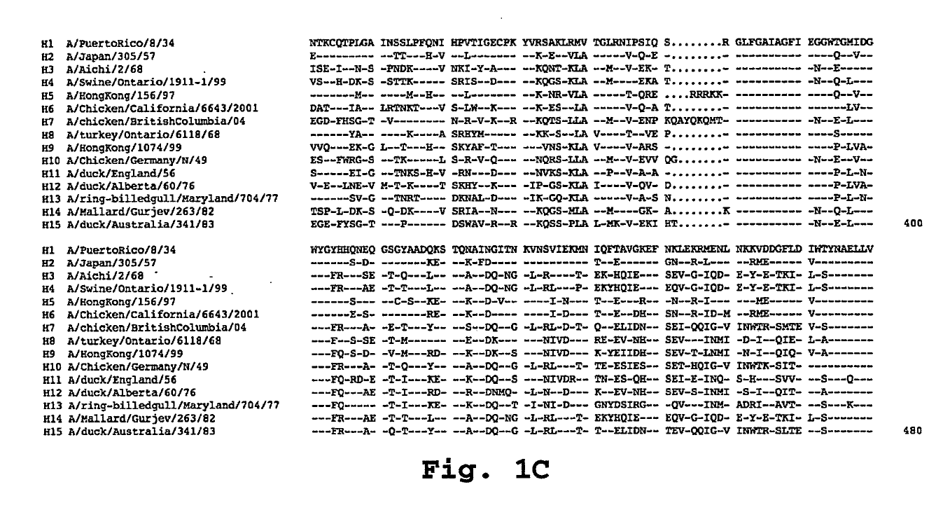

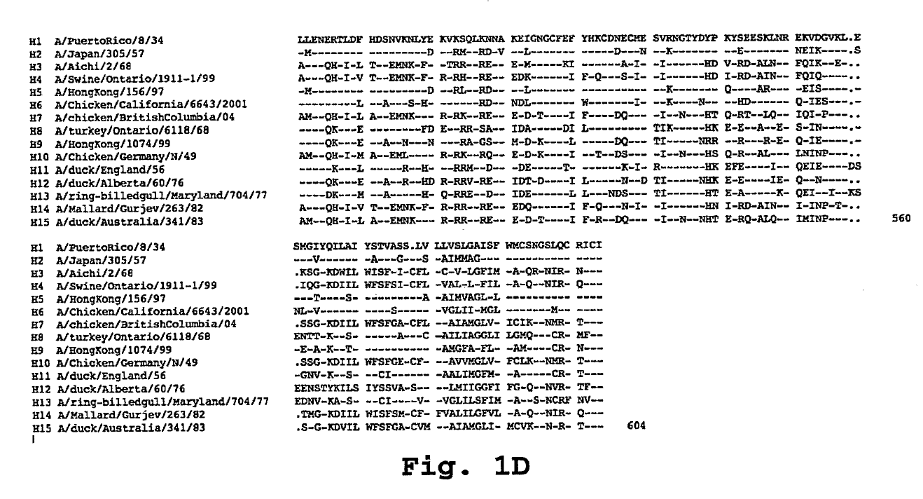

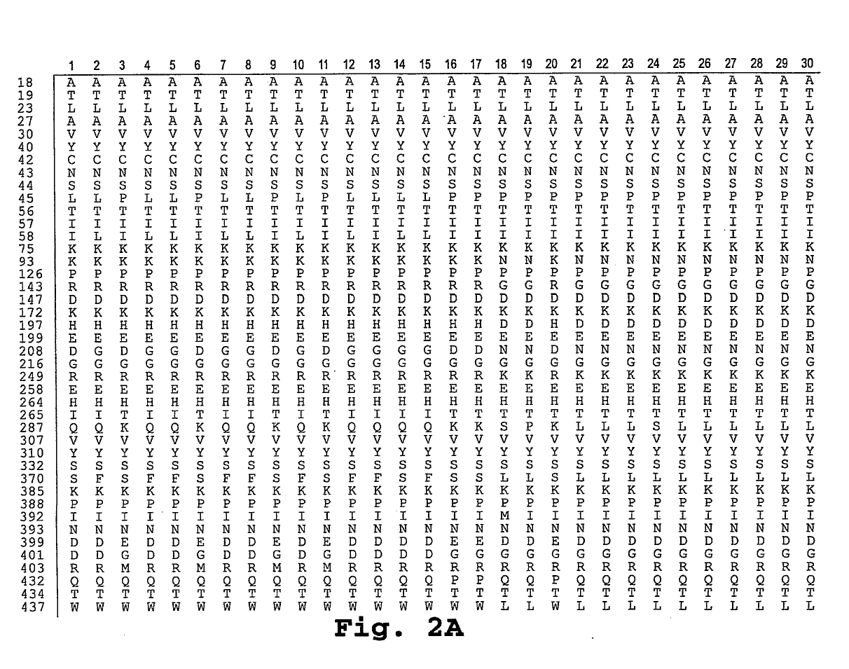

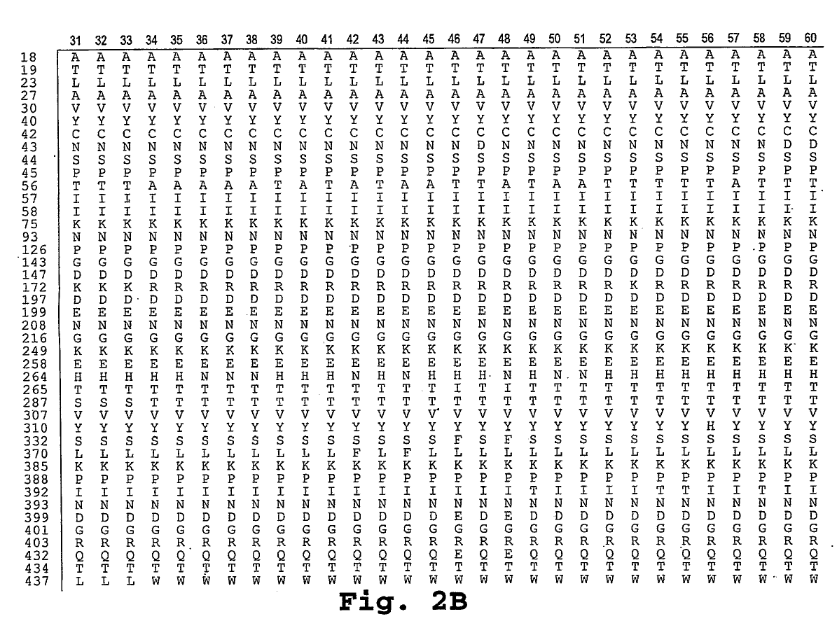

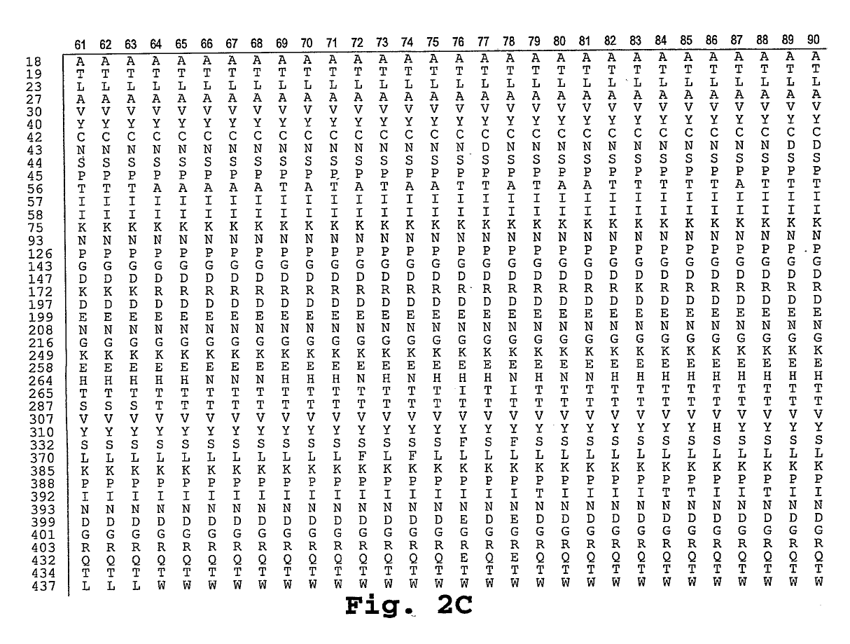

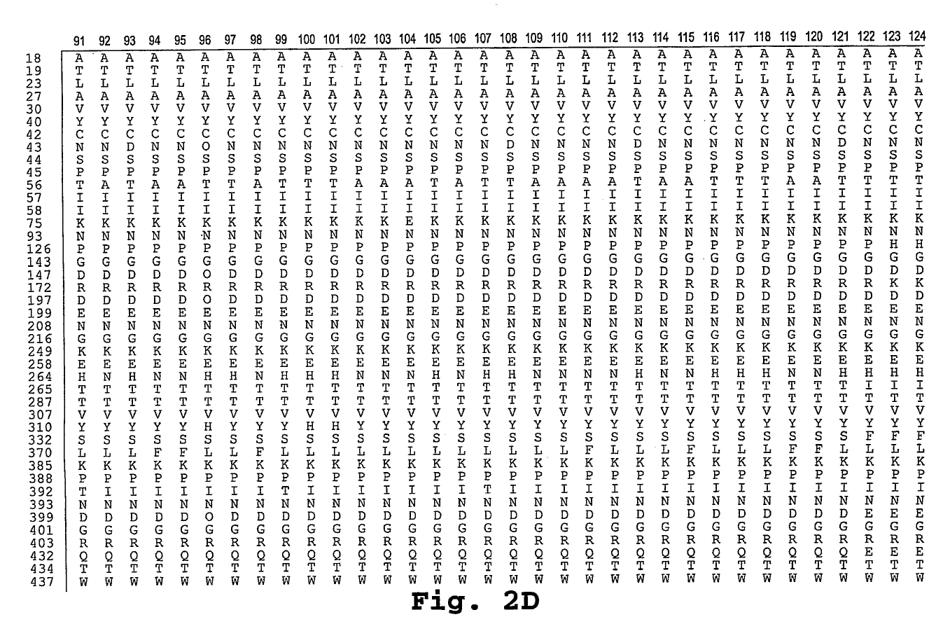

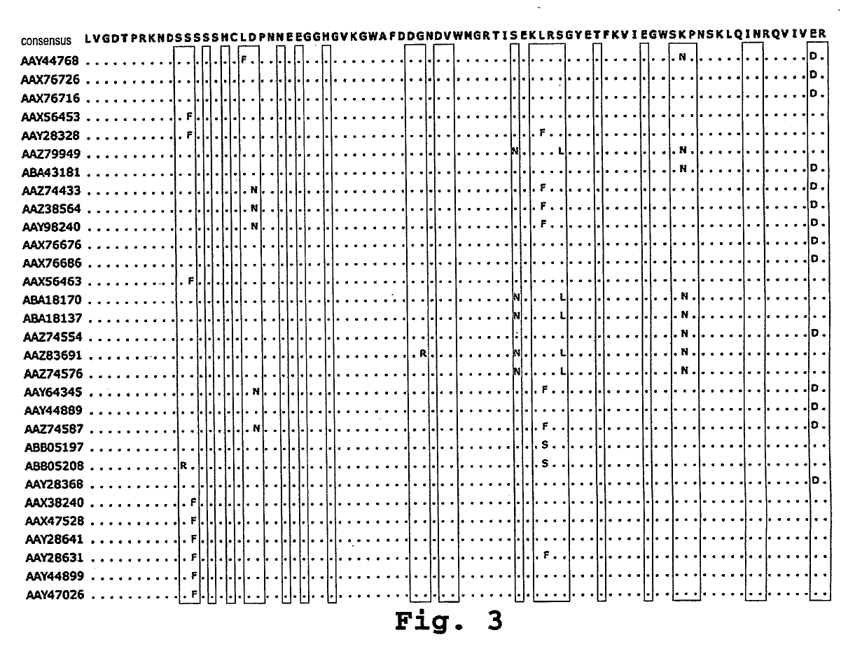

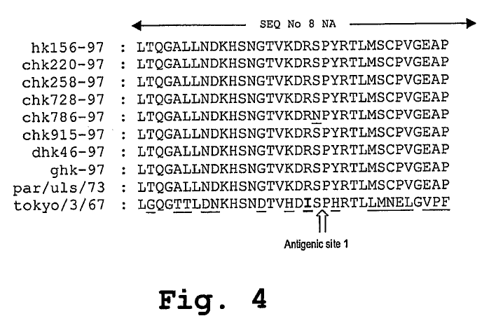

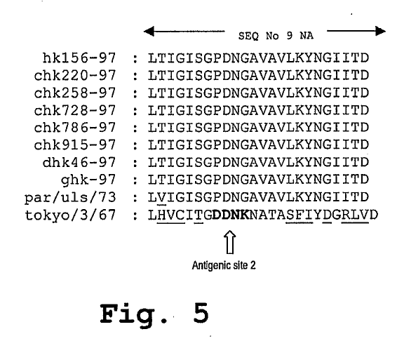

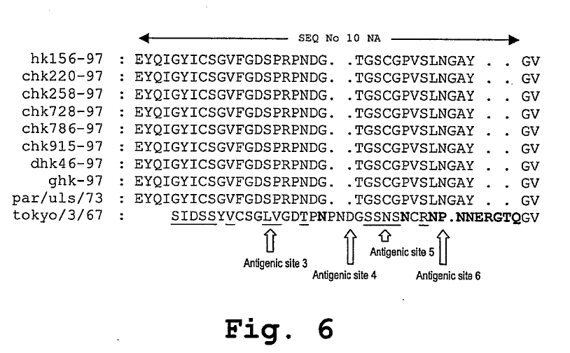

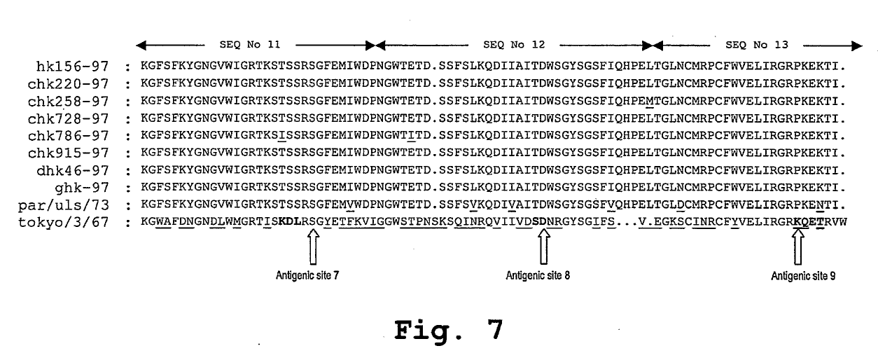

For the Influenza vaccine composition, a selected region of influenza A HA protein having high amino acid variability, corresponding to: 1) the “Receptor Binding Site”: Seq No 1 HA: (residues 211 to 240; including the 190-helix (residues 223-231) {See FIG. 1b}; Seq No 2 HA: (residues 151 to 180; including the 130-loop (residues 165-168) {See FIG. 1 and b}; Seq No 3 HA: (residues 151 to 180; including the 220-loop (residues 254-261) {See FIG. 1b}; and 2) the “cleavage site”, Seq No 4 HA: (residues 366 to 394; “Cleavage site” {residue 380} where for full infectivity, the single chain (HA0) is cut into two chains for full infectivity; and 3) {See FIG. 1c}; Seq No 5: NA (residues 18 to 437) (See FIG. 2 a-d); Seq No 6: NA (residues 321 to 341) {See FIG. 3}; and Seq No 7: NA (residues 342 to 400) {See FIG. 3}; Seq No 8: (residues 153 to 185), including antigenic site 1 (residue 170) {See FIG. 4}; Seq No 9: (residues 209 to 232), including antigenic site 2 (residues 216-219) {See FIG. 5}; Seq No 10: (residues 330 to 369), including antigenic site 3-4-5-6 (3=346, 4=353, 5=357-358, 6=361-367) {See FIG. 6}; Seq No 11: (residues 369 to 398), including antigenic site 7 (residues 387-388-389) {See FIG. 7}; Seq No 12: (residues 399 to 434), including antigenic site 8 (residues 420-421) {See FIG. 7}; Seq No 13: (residues 435 to 460), including antigenic site 9 (residues 454-457) {See FIG. 7}, the amino acid sequences from the influenza A HA protein for 15 known subtypes were aligned by sequence homology, as shown in FIG. 1. The presence or absence of amino acids from an aligned sequence of a particular variant is relative to a chosen consensus length of a reference sequence, which in the example shown in FIG. 1, were subtypes H1, H2, H3, H5, and H9 among the fifteen subtypes studied.

In order to maintain the highest homology in alignment of sequences, deletions in the sequence of a variant relative to the reference sequence can be represented by an amino acid space “-”, while insertional mutations in the variant relative to the reference sequence can be disregarded and left out of the sequence of the variant when aligned. Given N variants of the protein, the number of times that a given amino acid (aa) occurs at a given position n, the frequency of occurrence for that amino acid at that position n is calculated, as described in co-owned U.S. Pat. No. 6,432,675, which is incorporated herein by reference. The frequency at which an amino acid deletion occurs at a given position can be factored into this calculation as well.

Alternatively, if the deletions are not considered in the frequency calculation, then it may be desirable that the value of N used in the calculation at a given amino acid position n should be the number of variants less the number of variants in which an amino acid space is present at that given position. Based upon the determination of the frequency of occurrence of amino acid types at each position n in the population of variants, a “threshold value” for inclusion of a particular amino acid type at the corresponding position n for the set of polypeptide antigens is determined. A degenerate oligonucleotide sequence can then be created. The degenerate oligonucleotide sequence is designed to have the minimum number of nucleotide combinations necessary, at each codon position, to give rise to codons for each amino acid type selected based upon the chosen threshold value, as detailed in U.S. Pat. No. 6,432,675.

The threshold frequency used to select types of amino acids for inclusion in the set of polypeptide antigens and accordingly, for determining the degenerate oligonucleotide sequence, can be applied uniformly to each amino acid position. For instance, a threshold value of 15 percent can be applied across the entire protein sequence. Alternatively, the threshold value can be set for each amino acid position n independently. For example, the threshold value can be set at each amino acid position n so as to include the most commonly occurring amino acid types, e.g., those which appear at that position in at least 90% of the N variants.

It may, in some instances, be desirable to apply a further criterion to the determination of a degenerate oligonucleotide sequence which comprises restricting the degeneracy of a codon position such that no more than a given number of amino acid types can arise at the corresponding amino acid position in the set of polypeptide antigens. For example, the degenerate sequence of a given codon position n can be restricted such that selected amino acids will occur in at least about 11% of the polypeptides of the polypeptide antigen set. This means that all of the possible nucleotide combinations of that degenerate codon will give rise to no more than 9 different amino acids at the position. Thus, the frequency at which a particular amino acid appears at a given position will depend on the possible degeneracy of the corresponding codon position. Preferably, the number will be 11.1 (9 different amino acids), 12.5 (8 different amino acids), 16.6 (6 different amino acids), 25 (4 different amino acids) or 50 (2 different amino acids).

Likewise, criteria used for choosing the population of variants for frequency analysis can be determined by such factors as the expected utility of the polypeptide antigen set and factors concerning vaccination or tolerization. For example, analysis of a variant protein sequence can be restricted to subpopulations of a larger population of variants of the protein based on factors such as epidemiological data, including geographic occurrence or alternatively, on known allele families (such as variants of the DQ HLA class II allele). Likewise, in the case of protein components of pathogens, the population of variants selected for analysis can be chosen based on known tropisms for a particular susceptible host organism.

Applying this approach, the amino acid variants that occur in the influenza A HA region 91-160 for the influenza A subtypes shown in FIG. 1 were each examined for frequency of occurrence above a selected threshold level. The results of this analysis are shown in FIG. 2, where a specified residue represents an invariant position and “Xaa” represents one or more possible amino acid variations at that position. In preparing the composition of this invention, polypeptides representing each of the specified variants are preferably produced. More generally, the composition includes a majority of the possible sequence variations shown, preferably at least 70%, more preferably at least 80% of the sequence variations shown.

There are many ways by which the set of polypeptide antigens can be generated from the degenerate oligonucleotide sequence. Chemical synthesis of a degenerate oligonucleotide can be carried out in an automatic DNA synthesizer, and the synthetic oligonucleotides can then be ligated into an appropriate gene for expression. A start codon (ATG) can be engineered into the sequence if desired. The degenerate oligonucleotide sequences can be incorporated into a gene construct so as to allow expression of a protein consisting essentially of the set of polypeptide antigens. Alternatively, the set of polypeptide antigens can be expressed as parts of fusion proteins. The gene library created can be brought under appropriate transcriptional control by manipulation of transcriptional regulatory sequences. It may be desirable to create fusion proteins containing a leader sequence which directs transport of the recombinant proteins along appropriate cellular secretory routes.

Various methods of chemically synthesizing polydeoxynucleotides are known, including solid-phase synthesis which, like peptide synthesis, has been fully automated in commercially available DNA synthesizers (See the Itakura et al. U.S. Pat. No. 4,598,049; the Caruthers et al. U.S. Pat. No. 4,458,066; and the Itakura U.S. Pat. Nos. 4,401,796 and 4,373,071, incorporated by reference herein).

The purpose of a degenerate set of oligonucleotides is to provide, in one mixture, all of the sequences encoding the desired set of polypeptide antigens. It will generally not be practical to synthesize each oligonucleotide of this mixture one by one, particularly in the case of great numbers of possible variants. In these instances, the mixture can be synthesized by a strategy in which a mixture of coupling units (nucleotide monomers) are added at the appropriate positions in the sequence such that the final oligonucleotide mixture includes the sequences coding for the desired set of polypeptide antigens. Conventional techniques of DNA synthesis take advantage of protecting groups on the reactive deoxynucleotides such that, upon incorporation into a growing oligomer, further coupling to that oligomer is inhibited until a subsequent deprotecting step is provided. Thus, to create a degenerate sequence, more than one type of deoxynucleotide can be simultaneously reacted with the growing oligonucleotide during a round of coupling, either by premixing nucleotides or by programming the synthesizer to deliver appropriate volumes of nucleotide-containing reactant solutions. For each codon position corresponding to an amino acid position having only one amino acid type in the eventual set of polypeptide antigens, each oligonucleotide of the degenerate set of oligonucleotides will have an identical nucleotide sequence. At a codon position corresponding to an amino acid position at which more than one amino acid type will occur in the eventual set, the degenerate set of oligonucleotides will comprise nucleotide sequences giving rise to codons which code for those amino acid types at that position in the set. In some instances, due to other combinations that the degenerate nucleotide sequence can have, the resulting oligonucleotides will have codons directed to amino acid types other than those designed to be present based on analysis of the frequency of occurrence in the variant. The synthesis of degenerate oligonucleotides is well known in the art (see for example Narang, S A (1983) Tetrahedron 39:3; Itakura et al. (1981) in Recombinant DNA, Proc 3rd Cleveland Sympos. Macromolecules, ed. A G Walton, Amsterdam: Elsevier pp 273-289; Itakura at al. (1984) Annu. Rev. Biochem, 53:323; Itakura et al (1984) Science 198:1056; Ike et al. (1983) Nucleic Acid Res. 11:477, incorporated by reference herein).

As noted above, one strategy of synthesizing the degenerate oligonucleotide involves simultaneously reacting more than one type of deoxynucleotide during a given round of coupling. For instance, if either a Histidine (His) or Threonine (Thr) was to appear at a given amino acid position, the synthesis of the set of oligonucleotides could be carried out as follows: (assuming synthesis were proceeding 3′ to 5′) the growing oligonucleotide would first be coupled to a 5′-protected thymidine deoxynucleotide, deprotected, then simultaneously reacted with a mixture of a 5′-protected adenine deoxynucleotide and a 5′-protected cytidine deoxynucleotide. Upon deprotection of the resulting oligonucleotides, another mixture of a 5′-protected adenine deoxynucleotide and a 5′-protected cytidine deoxynucleotide are simultaneously reacted. The resulting set of oligonucleotides will contain at that codon position either ACT (Thr), AAT (Asn), CAT (His) or CCT (Pro). Thus, when more than one nucleotide of a codon is varied, the use of nucleotide monomers in the synthesis can potentially result in a mixture of codons including, but not limited to, those designed to be present by frequency analysis.

To create an amino acid space (deletion) at a given amino acid position, a portion of the oligonucleotide mixture can be held aside during the appropriate rounds of nucleotide additions (i.e., three coupling rounds per codon) so as to lack a particular codon position all together, then added back to the mixture at the start of synthesis of the subsequent codon position.

The entire coding sequence for the polypeptide antigen set can be synthesized by this method. In some instances, it may be desirable to synthesize degenerate oligonucleotide fragments by this method. Such fragments are then ligated to invariant DNA sequences synthesized separately to create a longer degenerate oligonucleotide.

Likewise, the amino acid positions containing more than one amino acid type in the generated set of polypeptide antigens need not be contiguous in the polypeptide sequence. In some instances, it may be desirable to synthesize a number of degenerate oligonucleotide fragments, each fragment corresponding to a distinct fragment of the coding sequence for the set of polypeptide antigens. Each degenerate oligonucleotide fragment can then be enzymatically ligated to the appropriate invariant DNA sequences coding for stretches of amino acids for which only one amino acid type occurs at each position in the set of polypeptide antigens. Thus, the final degenerate coding sequence is created by fusion of both degenerate and invariant sequences.

These methods are useful when the frequency-based mutations are concentrated in portions of the polypeptide antigen to be generated and it is desirable to synthesis long invariant nucleotide sequences separately from the synthesis of degenerate nucleotide sequences.

Furthermore, the degenerate oligonucleotide can be synthesized as degenerate fragments and ligated together (i.e., complementary overhangs can be created, or blunt-end ligation can be used). It is common to synthesize overlapping fragments as complementary strands, then annealing and filling in the remaining single-stranded regions of each strand. It will generally be desirable in instances requiring annealing of complementary strands that the junction be in an area of little degeneracy.

The nucleotide sequences derived from the synthesis of a degenerate oligonucleotide sequence and encoding the set of polypeptide antigens can be used to produce the set of polypeptide antigens via microbial processes. Ligating the sequences into a gene construct, such as an expression vector, and transforming or transfecting into hosts, either eukaryotic (yeast, avian or mammalian) or prokaryotic (bacterial cells), are standard procedures used in producing other well-known proteins, e.g. insulin, interferons, human growth hormone, IL-1, IL-2, and the like. Similar procedures, or obvious modifications thereof, can be employed to prepare the set of polypeptide antigens by microbial means or tissue-culture technology in accord with the subject invention.

As stated above, the degenerate set of oligonucleotides coding for the set of polypeptide antigens in the form of a library of gene constructs can be ligated into a vector suitable for expression in either prokaryotic cells, eukaryotic cells, or both. Expression vehicles for production of the set of polypeptide antigens of this invention include plasmids or other vectors. For instance, suitable vectors for the expression of the degenerate set of oligonucleotides include plasmids of the types: pBR322, pEMBL plasmids, pEX plasmids, pBTac plasmids and pUC plasmids for expression in prokaryotic cells, such as E. coli.

A number of vectors exist for the expression of recombinant proteins in yeast. For instance, YEP24, YIP5, YEP51, YEP52 and YRP17 are cloning and expression vehicles useful in the introduction of genetic constructs into S. cerevisiae (see for example Broach et al. (1983) in Experimental Manipulation of Gene Expression, ed M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. These vectors are modified with sequences from bacterial plasmids such as pBR322 to facilitate replication and drug resistance selection in both prokaryotic and eukaryotic cells. Alternatively, derivatives of viruses such as the bovine papilloma virus (BPV-1), Epstein-Barr virus (pHEBo and p205) can be used for transient expression of proteins in eukaryotic cells. The various methods employed in the preparation of the plasmids and transformation of host organisms are well known in the art. For other suitable expression systems for both prokaryotic and eukaryotic, as well as general recombinant procedures, see Molecular Cloning, 2nd Ed., ed. by Sambrook, Fritsch and Maniatis (Cold Spring Harbor Laboratory Press: 1989) incorporated by reference herein.

To express the library of gene constructs of the degenerate set of oligonucleotides, it may be desirable to include transcriptional and translational regulatory elements and other non-coding sequences to the expression construct. For instance, regulatory elements including constitutive and inducible promoters and enhancers can be incorporated.

In some instances, it will be necessary to add a start codon (ATG) to the degenerate oligonucleotide sequence. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine if desired can be achieved either in vivo by expressing the set of polypeptide antigens in a host which produces MAP (e.g., E. coli or CM89 or S. cerevisiae) or in vitro by use of Purified MAP (e.g., procedure of Miller et al.).

Alternatively, the coding sequences for the polypeptide antigens can be incorporated as a part of a fusion gene including an endogenous protein for expression by the microorganism. For example, the VP6 capsid protein of rotavirus can be used as an immunologic carrier protein for the polypeptide antigen set, either in the monomeric form or in the form of a viral particle. The set of degenerate oligonucleotide sequences can be incorporated into a fusion gene construct which includes coding sequences for a late vaccinia virus structural protein to produce a set of recombinant viruses expressing fusion proteins comprising the set of polypeptide antigens as part of the virion. It has been demonstrated with the use of V-3 loop/Hepatitis B surface antigen fusion proteins that recombinant Hepatitis B virions can be utilized in this role as well. Similarly, chimeric constructs coding for fusion proteins containing the set of polypeptide antigens and the poliovirus capsid protein can be created to enhance immunogenecity of the set of polypeptide antigens. The use of such fusion protein expression systems to establish a set of polypeptide antigens has the advantage that often both B-cell proliferation in response to the immunogen can be elicited. (see for example EP Publication No. 0259149; and Evans et al. (1989) Nature 339:385; Huang at al. (1988) J. Virol. 62:3855; and Schlienger et al. (1992) J. Virol. 66:2, incorporated by reference herein). The Multiple Antigen Peptide (MAP) system for peptide-based vaccines can be utilized in which the polypeptide antigen set is obtained directly from organo-chemical synthesis of the peptides onto an oligomeric branching lysine core (see for example Posnett et al. (1988) JBC 263:1719 and Nardelli et al. (1992) J. Immunol. 148:914, incorporated by reference herein). Foreign antigenic determinants can also be expressed and presented by bacterial cells.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Alternatively, the fusion gene can be synthesized by conventional techniques including automated DNA synthesizers.

An alternative approach to generating the set of polypeptide antigens is to carry out the peptide synthesis directly. At each codon position n in the degenerate oligonucleotide, each possible nucleotide combination can be determined and the corresponding amino acid designated for inclusion at the corresponding amino acid position of the polypeptide antigen set. Thus, synthesis of a degenerate polypeptide sequence can be directed in which sequence divergence occurs at those amino acid positions at which more than one amino acid is coded for in the corresponding codon position of the degenerate oligonucleotide. Organo-chemical synthesis of polypeptides is well known and can be carried out by procedures such as solid state peptide synthesis using automated protein synthesizers.

The synthesis of polypeptides is generally carried out through the Condensation of the carboxy group of an amino acid, and the amino group of another amino acid, to form a peptide bond. A sequence can be constructed by repeating the condensation of individual amino acid residues in stepwise elongation, in a manner analogous to the synthesis of oligonucleotides. In such condensations, the amino and carboxy groups that are not to participate in the reaction can be blocked with protecting groups which are readily introduced, stable to the condensation reactions and selectively removable from the completed peptide. Thus, the overall process generally comprises protection, activation, coupling and deprotection. If a peptide involves amino acids with side chains that may react during condensation, the side chains can also be reversibly protected, removable at the final stage of synthesis.

A successful synthesis for a large polypeptide by a linear strategy must achieve nearly quantitative recoveries for each chemical step. Many automated peptide synthesis schemes take advantage of attachment of the growing polypeptide chain to an insoluble polymer resin support such that the polypeptide can be washed free of byproducts and excess reactants after each reaction step (see for example Merrifield (1963) J.A.C.S. 85:2149; Chang et al. (1978) Int. J. Peptide Protein Res. 11:246; Barany and Merrifield, The Peptides, vol 2 .COPYRGT.1979 NY: Academic Press, pp 1-284; Tam, J. P. (1988) PNAS 85:5409; and Tam et al. U.S. Pat. No. 4,507,230, incorporated herein in its entirety by reference). For example, a first amino acid is attached to a resin by a cleavable linkage to its carboxylic group, deblocked at its amino acid side, and coupled with a second activated amino acid carrying a protected .alpha.-amino group. The resulting protected dipeptide is deblocked to yield a free amino terminus, and coupled to a third N-protected amino acid. After many repetitions of these steps, the complete polypeptide is cleaved from the resin support and appropriately deprotected.

To generate the set of polypeptide antigens, more than one N-protected amino acid type can be reacted simultaneously in each round of coupling with the growing polypeptide chain to create the desired degenerate amino acid sequence at each amino acid position. In one embodiment, the set of polypeptides will include only those amino acids that are present at any position n in the population of variants above the predetermined threshold frequency.

Alternatively, one can first design the degenerate oligonucleotide, determine the amino acids encoded by the combination of codons and include all the amino acids in the chemical synthesis. For example, a degenerate codon at codon position n, having the sequence MMT and thus coding for either a Thr (ACT), an Asn (AAT), a His (CAT) or a Pro (CCT), can be created at the peptide synthesis level by reacting all four N-protected amino acid types simultaneously with the free amino terminus of the growing, resin-bound peptide. Thus, four subpopulations of peptides will be created, each subpopulation definable by the amino acid type present at the amino acid position n corresponding to the codon position n.

Because the amino acid being added to the resin-bound polypeptide is protected, the growth of the peptide chain is terminated upon addition of the protected amino acid until the subsequent deblocking step. Those skilled in the art will recognize that, due to potential differences in reactivity of various amino acid analogs, it may be desirable to use non-equimolar ratios of amino acid types when simultaneously reacting more than one amino acid type in order to get equimolar ratios of subpopulations. Alternatively, it may be desirable to divide the resin-bound polypeptide into aliquots, each of which is reacted with a distinct amino acid type, the polypeptide products being recombined prior to the next coupling reaction. This technique can be applied to create an amino acid gap in a subpopulation, simply by holding aside an appropriate aliquot during one round of coupling, then recombining all resin-bound polypeptides prior to the next round of coupling. Furthermore, it is apparent that, from the many different blocking and activating groups available, chemical synthesis of the polypeptide can be carried out in either the N-terminal to C-terminal, or C-terminal to N-terminal direction.

The generated set of polypeptide antigens can be covalently or noncovalently modified with non-proteinaceous materials such as lipids or carbohydrates to enhance immunogenecity or solubility. The present invention is understood to include all such chemical modifications of the set of polypeptide antigens so long as the modified peptide antigens retain substantially all the antigenic/immunogenic properties of the parent mixture.

The generated set of polypeptide antigens can also be coupled with or incorporated into a viral particle, a replicating virus, or other microorganism in order to enhance immunogenicity. The set of polypeptide antigens may be chemically attached to the viral particle or microorganism or an immunogenic portion thereof.

There are a large number of chemical cross-linking agents that are known to those skilled in the art. For the present invention, the preferred cross-linking agents are heterobifunctional cross-linkers, which can be used to link proteins in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art. These include: succinimidyl 4-(N-maleimidomethyl)cyclohexane-1-carboxylate (SMCC), m-Maleimidobenzoyl-N-hydroxysuccinimide ester (MBS); N-succinimidyl (4-iodoacetyl)aminobenzoate (SIAB), succinimidyl 4-(p-maleimidophenyl)butyrate (SMPB), 1-ethyl-3-(3-dimethylaminopropyl) carbodiimide hydrochloride (EDC), 4-succinimidyloxycarbonyl-.alpha.-methyl-.alpha.-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

The introduction of antigen into an animal initiates a series of events culminating in both cellular and humoral immunity. By convention, the property of a molecule that allows it to induce an immune response is called immunogenicity. The property of being able to react with an antibody that has been induced is called antigenicity. Antibodies able to cross-react with two or more different antigens can do so by virtue of some degree of structural and chemical similarity between the antigenic determinants (or “epitopes”) of the antigens. A protein immunogen is usually composed of a number of antigenic determinants. Hence, immunizing with a protein results in the formation of antibody molecules with different specificities, the number of different antibodies depending on the number of antigenic determinants and their inherent immunogenicity.

Proteins are highly immunogenic when injected into an animal for which they are not normal (“self”) constituents. Conversely, peptides and other compounds with molecular weights below about 5000 (termed “haptens”) daltons, by themselves, do not generally elicit the formation of antibodies. However, if these small molecule antigens are first coupled with a longer immunogenic antigen such as a protein, antibodies can be raised which specifically bind epitopes on the small molecules. Conjugation of haptens to carrier proteins can be carried out as described above.

When necessary, modification of such ligand to prepare an immunogen should take into account the effect on the structural specificity of the antibody. That is, in choosing a site on a ligand for conjugation to a carrier such as protein, the selected site is chosen so that administration of the resulting immunogen will provide antibodies which will recognize the original ligand. Furthermore, not only must the antibody recognize the original ligand, but significant characteristics of the ligand portion of the immunogen must remain so that the antibody produced after administration of the immunogen will more likely distinguish compounds closely related to the ligand which may also be present in the patient sample. In addition, the antibodies should have high binding constants.

Vaccines comprising the generated set of polypeptide antigens, and variants thereof having antigenic properties, can be prepared by procedures well known in the art. For example, such vaccines can be prepared as injectables, e.g., liquid solutions or suspensions. Solid forms for solution in, or suspension in, a liquid prior to injection also can be prepared. Optionally, the preparation also can be emulsified. The active antigenic ingredient or ingredients can be mixed with excipients which are pharmaceutically acceptable and compatible with the active ingredient. Examples of suitable excipients are water, saline, dextrose, glycerol, ethanol, or the like, and combinations thereof. In addition, if desired, the vaccine can contain minor amounts of auxiliary substances such as wetting or emulsifying agents, pH buffering agents, or adjuvants such as aluminum hydroxide or muramyl dipeptide or variations thereof. In the case of peptides, coupling to larger molecules such as Keyhole limpet hemacyanin (KLH) sometimes enhances immunogenicity. The vaccines are conventionally administered parenterally, by injection, for example, either subcutaneously or intramuscularly. Additional formulations which are suitable for other modes of administration include suppositories and, in some cases, oral formulations. For suppositories, the traditional binders and carriers include, for example, polyalkalene glycols or triglycerides. Suppositories can be formed from mixtures containing the active ingredient in the range of about 0.5% to about 10%, preferably about 1% to about 2%. Oral formulations can include such normally employed excipients as, for example, pharmaceutical grades of mannitol, lactose, starch, magnesium stearate, sodium saccharine, cellulose, magnesium carbonate, and the like. These compositions can take the form of solutions, suspensions, tablets, pills, capsules, sustained release formulations or powders and contain from about 10% to about 95% of active ingredient, preferably from about 25% to about 70%.

The active compounds can be formulated into the vaccine as neutral or salt forms. Pharmaceutically acceptable salts include the acid addition salts (formed with the free amino groups of the polypeptides) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Viruses contain many molecules that are distinguished as being foreign to the body. Their antigens, or epitopes are specifically recognized by the B cell and T cell receptors and results in cellular activation. Each individual T cell or B cell will only recognize and respond to its individual cognate “epitope”. Once activated, activated CD8+ CTL T cells will attack and destroy cells infected by the invading virus. Other CD4+ T cell (Th) or B cell may respond by making many duplicate copies of itself and remain in the body as memory cells. If the body is re-invaded by the virus in the future, these memory cells will be reactivated and respond faster and more powerfully to destroy the virus. This is the principle behind vaccines, such as the vaccinations we received in childhood against measles or mumps.

T cells recognize epitopes displayed in the context of major histocompatibility complexes (MHC, also known as HLA for Human Leoocyte antigens) via their T cell receptors. The division of CD8+ T cells and CD4+ T cells also engage different type MHC complexes. CD8+ T cells recognize epitopes in the context of MHC class I molecules, whereas CD4+ T cells recognize peptide-antigens in the context of MHC class II. As stated above, CD4+ and CD8+ T cells differ in their immune responses. CD4+ T mediated is more complex, by providing help via cytokine production to other immune system components namely, B-cells and/or CD8+ T cells. On the other hand, CD8+ T cell is simpler as these CTLs directly destroy cells expressing MHC class I complexes with the foreign epitope. Therefore, cytotoxic CD8+ T lymphocytes (CTL)-mediated immune responses play a central role in protective immunity against many viral and intracellular bacterial infections.

Another important factor is the ability of the cellular antigen processing machinery to generate a certain peptide-MHC complex by the antigen presenting cell (APC). Many molecules have been identified that participate in the process of antigen presentation including the proteasome, a multicatalytic protease and TAP (transporters associated with antigen processing) molecules. Antigen processing events appear to have peptide-dependent activity, which bias certain amino acid residues and sequences for presentation on MHC I and MHC II. Therefore is it important to identify binding epitopes that elicit T-cell responses in humans. Some assays to test T-cell responses after in vitro stimulation include: cytotoxicity assays, proliferation assays, cytokine measurements, flow cytometry analyses.

A vaccine composition may include peptides containing a cocktail of multipeptide CD8 T and CD4 T helper cell focused epitopes in combination with protein fragments containing the principal neutralizing domain. For instance, several of these epitopes have been mapped within the HIV envelope, and these regions have been shown to stimulate proliferation and lymphokine release from lymphocytes. Providing both of these epitopes in a vaccine comprising a generated set of polypeptide antigens derived from analysis of HIV-1 isolates can result in the stimulation of both the humoral and the cellular immune responses. In addition, commercial carriers and adjuvants are available to enhance immunomodulation of both B-cell and T-cell populations for an immunogen (for example, the IMJECT SUPERCARRIER™ System, Pierce Chemical, Catalog No. 77151G).

Alternatively, a vaccine composition may include a compound which functions to increase the general immune response. One such compound is interleukin-2 (IL-2) which has been reported to enhance immunogenicity by general immune stimulation (Nunberg et al. (1988) In New Chemical and Genetic Approaches to Vaccination, Cold Spring Harbor Laboratory, Cold Spring Harbor, N.Y.). IL-2 may be coupled the polypeptides of the generated set of polypeptide antigens to enhance the efficacy of vaccination.

The vaccines are administered in a manner compatible with the dosage formulation, and in such amount as will be therapeutically effective and immunogenic. The quantity to be administered depends on the subject to be treated, capacity of the subject's immune system to synthesize antibodies, and the degree of protection desired. Precise amounts of active ingredient required to be administered depend on the judgment of the practitioner and are peculiar to each individual. Suitable regimes for initial administration and booster shots are also variable, but are typified by an initial administration followed in one or two week intervals by a subsequent injection or other administration.

In preparing a combinatorial peptide antigen vaccine, there is a practical limit to the number of different epitopes that can be made. A balance in minimizing the number of unique antigens while maximizing the breadth of their reactivity must be reached. Rational design and feasibility of the multivalent polypeptide vaccines will centers on which of the many possible combinatorial epitope candidates should be included to elicit broadly effective immune responses. Influenza strains worldwide display tremendous genetic diversity. Some regions of the protein are conserved while other portions have differing degrees of sequence conservation which could produce very high numbers of combinatorial sequences. To illustrate this point see Seq No2 HA, where there are 55,296 distinct sequence combinations that could be included as part of a vaccine composition. Inclusion of all 55,296 discrete polypeptides in a vaccine composition may not cause sufficient immunological responses as each individual peptide will likely be introduced in minute amounts. To be more effective, a limited subset of the available 55 296 Seq No2 HA sequences can be chosen to increase the available protein concentration of each individual peptide. This collection of peptides could range from as low as a single peptide sequence up to one hundred different sequences. The advantage of incorporating multiple epitopes is to elicit immune responses against multiple influenza strains.

One approach to limit the combinatorial variability at some positions, in the identified epitopes Seq No 1 to 13, is to determine which of available amino acids are most commonly associated with influenza strains that can infect humans. Of the influenza type A viruses, only the subtypes H1, H2 and H3 are easily transmitted between humans. There are recent cases where the H5N1 subtype has crossed over from avian hosts, such as chickens, to infect and cause limited outbreaks in humans. Other influenza A subtypes such as H7 and H9 are also known to sporadically infect humans. From FIG. 1, it can be seen that the amino acids E, D, S, T or N may occur at the third variable (Aa₃) position of Seq No2 HA between influenza subtypes H1 to H15. However for inclusion into our combinatorial Seq No2 HA vaccine design, we would only choose E, D or S as these are the amino acids in human influenza subtypes H1, H2, H3, H7 and H9.

Examining the genetic variation present from human derived geographic subtype isolates can be another method to minimize combinatorial diversity. Geographic isolation of certain genetic subtypes may prompt a customized local polypeptide vaccine in contrast to trying to deal with the extensive diversity worldwide influenza strains. The regional vaccine design would protect the resident population against those and genetically similar viruses circulating in that geographic locale. The genetic diversity at the identified epitopes (Seq No 1-13) would still permit construction of novel combinatorial sequences that offer greater immunological breadth in reactivity than the original isolates. Example 1a below demonstrates this procedure where five H5N1 influenza human isolates were recovered in Thailand (Puthavathana et al., Journal of General Virology, 2005. 85:423-433). The HA sequences were aligned to identify the which positions and what amino acids were variable in each of the Seq No 1-13 epitopes.

Examining the Seq No2 HA alignment, it can be seen that there were only four variable positions and all other amino acids were conserved. Seq No:2 HA has the potential to be varied at twelve positions along this epitope. However, the twenty-eight identified conserved amino acids between the five That H5N1 human isolates would not be further substituted in the subsequent combinatorial vaccine design. This conservation would then tend to increase the potential immunological reactivity by increasing the sequence similarity between the vaccine epitopes and local circulating H5N1 viral population. Uninfected people who are then vaccinated and will have sufficient cross-protective immune responses to any one circulating strains from the same H5N1 subtype especially if one became the epidemic dominant subtype.

Another approach to further minimize the amount of combinatorial sequences would be predicting which amino acid(s) the variable positions would most likely drift to. The variable positions in aligned epitopes would be enumerated for frequency of amino acid occurrence. The evolutionary trajectory of any one variable position would be toward the more frequent amino acid(s) at that position as it is a known tolerated change. In example 1b, the third variable amino acid position of Seq No2 HA of strain 286H is an S (serine). Comparing the other human H5N1 isolates, we find that L (leucine) predominates in four of the six strains. Thus the most likely probabilistic change for the 286H at the third position is from a S (serine) to a L (leucine) giving rise to new possible 286H variant 1.

A further method to limit combinatorial possibilities is to select, in some cases, those combinatorial sequences with only one amino acid change from a starting wild type epitope sequence. Mutational drift of proteins would likely experience one amino acid change at a time as evolutionary pressure will select for sequences on the basis of advantageous functional characteristics. Too great a change from the parental wild type epitope sequence may cause protein misfolding and/or loss of function. Therefore we would incorporate novel sequence combinations that show only one amino acid divergence from the starting parental wild type sequence. These new combinations would tend to be most similar to the majority of contemporary circulating sequences. In this manner we would not generate a spectrum of evolutionarily “distant” derivatives differing by five amino acids from any of the six starting wild-type epitopes.

In summary, where the potential number of combinatorial peptide antigens is quite large, as in the case of DES ID NOS: 1-13, it is necessary to further limit the amino acids to one or more of (i) particular influenza viral subtypes, (ii) strains within a particular geographic region (for vaccinating a population within that region), and (iii) limiting combinatorial variants to one single amino acid changes with respect to any known variant sequence. The total number of peptide antigens in the vaccine is preferably between 5 and 100, more preferably 5 and 50, where each of the different peptide antigens is present in amounts sufficient to produce an immune response in the vaccinated subject. Typically, the peptide antigens making up the vaccine composition are present in roughly equal-molar or equal-weigh amounts. An example of the method for selecting a suitable-number peptide antigen vaccine from the combinatorial peptides given by SEQ ID NO: 2 is given in Examples 1a and 1b below.

Antigens that induce tolerance are called toleragens, to be distinguished from immunogens, which generate immunity. Exposure of an individual to immunogenic antigens stimulates specific immunity, and for most immunogenic proteins, subsequent exposures generate enhanced Secondary responses. In contrast, exposure to a toleragenic antigen not only fails to induce specific immunity, but also inhibits lymphocyte activation by subsequent administration of immunogenic forms of the same antigen. Many foreign antigens can be immunogens or toleragens, depending on the physicochemical form, dose, and route of administration. This ability to manipulate responses to antigens can be exploited clinically to augment or suppress specific immunity. For instance, it can be desirable in the context of organ transplant technology to tolerize a transplant recipient with a polypeptide antigen set derived from the frequency analysis of known sub-haplotypes of a class II peptide (i.e., such as the DQ or DR allele products) present on the transplanted tissue in order to minimize rejection. It is also within the equivalence of this invention that the set of polypeptide antigens can be chemically coupled or incorporated as part of a fusion protein with an apoptotic agent, for instance an agent which brings about deregulation of C-myc expression or a cell toxin such as diphtheria toxoid, such that programmed cell death is brought about in an antigen specific manner.

Thus it would be routine for one skilled in the art to determine the appropriate administration regimen necessary to induce tolerance to the set of polypeptide antigens of the present invention.

### EXAMPLES

### Example 1a

This invention describes modifications in the peptide or DNA sequences that can be made by those skilled in the art using known techniques. In particular, the modifications of interest in the polypeptide sequences include the introduction of selected amino acid(s) at predetermined sites. For example, in Seq No 2 HA; the reference wild type sequence for Influenza A strain Puerto Rico 8/34 is:

PKESSWPNHNTTKGVTAACS*HAGKSSFYR

This represent residues 151 to 180 of the HA protein. After alignment of various Influenza strains (H1 to H15) and determination of the amino acids that occur above the threshold value [with emphasis of the amino acids from H1, H2, H3 and H5], the following degenerate peptide sequences was derived (see Seq No 2 HA):

Aa₁ Aa₂Aa₃ Aa₄Aa₅ Aa₆Aa₇Aa₈ Aa₉Aa₁₀Aa₁₁ Aa₁₂Aa₁₃GAa₁₄Aa₁₅ Aa₁₆AC(*)Aa₁₇ Aa₁₈Aa₁₉Aa₂₀ Aa₂₁FAa₂₂Aa₂₃

Furthermore, by substituting in, identified threshold amino acids at each amino acid position, the resulting degenerate polypeptide(s) is then:

P₁ K₂ (S/T)₃ S₄ (S/T/R)₅ W₆ S₇ (G/N)₈ (V/H)₉ (T/K/N)₁₀ T₁₁ (T/N/S/D)₁₂ (V/S/A/N)₁₃ G (T/V)₁₄ (S/T)₁₅ (S/K/R)₁₆A C(*) S₁₇ R₁₈ G₁₉ G₂₀ G₂₁F S₂₂ S₂₃ (Y/F)₂₄ (R/S)₂₅

The total possible permutations of Seq No2 HA in this case exceed fifty five thousand (5.5×10⁴) different sequences. However, one may choose to make only a limited subset from the available combinations. First, it may not be economically practical to synthesize all fifty five thousand peptides for inclusion into a vaccine candidate. Second, the individual peptides may have to be introduced at a minimum effective amount when delivered as exogenous peptides. To generate specific immune response against a certain Seq No2 HA sequence, that polypeptide antigen may have to be administered between 5 to 50 μg in order to be effective. Administering fifty five thousand polypeptides at a concentration of 50 μg each would require at least 0.275 g of total protein in the vaccine composition that may be challenging to inject. Finally, some of the possible permutations may comprise such novel polypeptide sequences that they would be quite dissimilar to Seq No2 HA and would not generate anti-influenza HA responses.

Therefore it would be desirable to reduce the number of Seq No2 HA variants that need to be synthesized. This allows immunological focus on a discrete set of protein sequence and higher opportunity to achieve the desired immune response. Targeting a limited set of sequences would be desirable if the influenza strains to be targeted are known. In this manner, the generated immunological responses would be much more focused. For example, from a recent outbreak in Thailand of avian H5N1 influenza, the viral genomes derived from human isolates were sequence characterized (Puthavathana et al., Journal of General Virology, 2005. 85:423-433). It can be appreciated that the various H5N1 human derived isolates are very closely related to each other as their genomes display a high degree of sequence conservation. The complete HA polypeptide sequences of these H5N1 strains were deposited into the Los Alamos Influenza database (www.flu.lanl.gov) and are shown below with their respective Seq No2 HA in bold and underlined.

A/Thailand/NK165/2005

(SEQ ID NO: 15)

RIVLLFAIVSLVKSDQICIGYHANNSTEQVDTIMEKNVTVTHAQDILEKT
HNGKLCDLDGVKPLILRDCSVAGWLLGNPMCDEFINVPEWSYIVEKPNPA
NDLCYPGDFNDYEELKHLLSRINHFEKIQIIPKSSWSSHEASVGVSSACP
YQGKSSFFRNVVWLIKKNSTYPTIKRSYNNTNQEDLLVLWGIHHPNDAAE
QTKLYQNPTTYISVGTSTLNQRLVPRIATRSKVNGQSGRMEFFWTILKPN
DAINFESNGNFIAPEYAYKIVKKGDSTIMKSELEYGNCNTKCQTPMGAIN
SSMPFHNIHPLTIGECPKYVKSNRLVLATGLRNSPQREKRRKKRGLFGAI
AGFIEGGWQGMVDGWYGYHHSNEQGSGYAADKESTQKAIDGVTNKVNSII
DKMNTQFEAVGREFNNLERRIENLNKKMEDGFLDVWTYNAELLVLMENER
TLDFHDSNVKNLYDKVRLQLRDNAKELGNGCFEFYHKCDNECMESVRNGT
YDYPQYSEEARLKREEISGVKLESIGIYQILSIYSTVASSLALAIMVAGL
SLWMCSNGSLQCRICIKLESDX
A/Thailand/2-SP-33/2004

(SEQ ID NO: 16)

KIVLLFAIVSLVKSDQICIGYHANNSTEQVDTIMEKNVTVTHAQDILEKT
HNGKLCDLDGVKPLILRDCSVAGWLLGNPMCDEFINVPEWSYIVEKANPV
NDLCYPGDFNDYEELKHLLSRINHFEKIQIIPKSSWSSHEVSLGVSSACP
YQGKSSFFRNVVWLIKKNSTYPTIKRSYNNTNQEDLLVLWGIHHPNDAAE
QTKLYQNPTTYISVGTSTLNQRLVPRIATRSKVNGQSGRMEFFWTILKPN
DAINFESNGNFIAPEYAYKIVKKGDSTIMKSELEYGNCNTKCQTPMGAIN
SSMPFHNIHPLTIGECPKYVKSNRLVLATGLRNSPQRERRRKKRGLFGAI
AGFIEGGWQGMVDGWYGYHHSNEQGSGYAAAKESTQKAIDGVTNKVNSII
DKMNTQFEAVGREFNNLERRIENLNKKMEDGFLDVWTYNAELLVLMENER
TLDFHDSNVKNLYDKVRLQLKDNAKELGNGCFEFYHKCDNECMESVRNGT
YDYPQYSEEARLKREEISGVKLESIGIYQILSIYSTVASSLALAIMVAGL
SLWMCSNGSLQCRICI*ICEFRL*X
A/Thailand/Chaiyaphum/622/2004

(SEQ ID NO: 17)

KIVLLFAMVSLVKSDQICIGYHANNSTEQVDTIMEKNVTVTHAQDILEKT
HNGKLCDLDGVKPLILRDCSVAGWLLGNPMCDEFINVPEWSYIVEKANPV
NDLCYPGDFNDYEELKHLLSRINHFEKIQIIPKSSWSSHEASLGVSSACP
YQGRSSFFRNVVWLIKKNSTYPTIKRSYNNTNQEDLLVLWGIHHPNDAAE
QTKLYQNPTTYISVGTSTLNQRLVPRIATRSKVNGQSGRMEFFWTILKPN
DAINFESNGNFIAPEYAYKIVKKGDSTIMKSELEYGNCNTKCQTPMGAIN
SSMPFHNIHPLTIGECPKYVKSNRLVLATGLRNSPQRERRRKKRGLFGAI
AGFIEGGWQGMVDGWYGYHHSNEQGSGYAADKESTQKAIDGVTNKVNSII
DKMNTQFEAVGREFNNLERRIENLNKKMEDGFLDVWTYNAELLVLMENER
TLDFHDSNVKNLYDKVRLQLRDNAKELGNGCFEFYHKCDNECMESVRNGT
YDYPQYSEEARLKREEIGGVKLESIGIYQILSIYSTVASSLALAIMVAGL
SLWMCSNGSLQCRICI*ICEFRL*LX
A/Thailand/SP83/2004

(SEQ ID NO: 18)

KIVLLFAIVSLVKSDQICIGYHANNSTEQVDTIMEKNVTVTHAQDILEKT
HNGKLCDLDGVKPLILRDCSVAGWLLGNPMCDEFINVPEWSYIVEKANPV
NDLCYPGDFNDYEELKHLLSRINHFEKIQIIPKSSWSSHEASLGVSSACP
YQGKSSFFRNVVWLIKKNSTYPTIKRSYNNTNQEDLLVLWGIHHPNDAAE
QTKLYQNPTTYISVGTSTLNQRLVPRIATRSKVNGQSGRMEFFWTILKPN
DAINFESNGNFIAPEYAYKIVKKGDSTIMKSELEYGNCNTKCQTPMGAIN
SSMPFHNIHPLTIGECPKYVKSNRLVLATGLRNSPQRERRRKKRGLFGAI
AGFIEGGWQGMVDGWYGYHHSNEQGSGYAADKESTQKAIDGVTNKVNSII
DKMNTQFEAVGREFNNLERRIENLNKKMEDGFLDVWTYNAELLVLMENER
TLDFHDSNVKNLYDKVRLQLRDNAKELGNGCFEFYHKCDNECMESVRNGT
YDYPQYSEEARLKREEISGVKLESIGIYQILSIYSTVASSLALAIMVAGL
SLWMCSNGSLQCRICI*ICEFRL*LX
A/Thailand/1-KAN-1/2004

(SEQ ID NO: 19)

KIVLLFAIVSLVKSDQICIGYHANNSTEQVDTIMEKNVTVTHAQDILEKT
HNGKLCDLDGVKPLILRDCSVAGWLLGNPMCDEFINVPEWSYIVEKANPV
NDLCYPGDFNDYEELKHLLSRINHFEKIQIIPKSSWSSHEASLGVSSACP
YQRKSSFFRNVVWLIKKNSTYPTIKRSYNNTNQEDLLVLWGIHHPKDAAE
QTKLYQNPTTYISVGTSTLNQRLVPRIATRSKVNGQSGRMEFFWTILKPN
DAINFESNGNFIAPEYAYKIVKKGDSTIMKSELEYGNCNTKCQTPMGAIN
SSMPFHNIHPLTIGECPKYVKSNRLVLATGLRNSPQRERRRKKRGLFGAI
AGFIEGGWQGMVDGWYGTTHSNEQGSGYAADKESTQKAIDGVTNKVNSII
DKMNTQFEAVGREFNNLERRIENLNKKMEDGFLDVWTYNAELLVLMENER
TLDFHDSNVKNLYDKVRLQLRDNAKELGNGCFEFYHKCDNECMESVRNGT
YDYPQYSEEARLKREEISGVKLESIGIYQILSIYSTVASSLALAIMVAGL
SLWMCSNGSLQCRICIKICESRLRX

After alignment of their respective Seq No2 HA portions, identical amino acids are indicated by lowercase letters and variable amino acids indicated by bold uppercase at each position:

| | |
|---|---|
| NK165 | |
| pksswssheAsVgvssacpyqGKssffr | (SEQ ID NO:20) |
| 2-SP-33 | |
| pksswssheVsLgvssacpyqGKssffr | (SEQ ID NO:20) |
| Chaiyaphum/622 | |
| pksswssheAsLgvssacpyqGRssffr | (SEQ ID NO:20) |
| SP83: | |
| pksswssheAsLgvssacpyqGKssffr | (SEQ ID NO:20) |
| 1-KAN-1: | |
| pksswssheAsLgvssacpyqRKssffr | (SEQ ID NO:20) |

Thus, to protect the local That population and surrounding Asian areas, it would likely be more advantageous to immunize them against the current circulating strains and close sequence variants rather than all fifty five thousand Seq No2 HA permutations from the other H1 to H15 derivatives. From the above sequence analysis it is likely the evolutionary drift occurs by accumulating single amino acid substitutions. The above That isolates show only two amino acid differences between their Seq No2 HA sequences. After enumerating the variable position amino acids for combinatorial design, the degenerate Seq No2 HA sequence generated from these would be:

P K S S W S S H E (A/V) S (V/L) G V S S A C P Y G (R/G) (R/K) S S F F R (SEQ ID NO: 20), yielding sixteen distinct Seq No2 HA possibilities. Five Seq No2 HA sequences represent the original wild type isolates and eleven are novel combinatorial derivatives. The Seq No2 HA alignment above also demonstrates which positions are most likely to undergo mutational drift and toward which other amino acids. In examining the five H5N1 isolates, most of the Seq No2 HA amino acid positions have been conserved except at four locations. The amino acids occurring at Seq No2 HA position 10 are A (alanine) and V (valine) both of which are aliphatic residues. Hence, Seq No2 HA position 10 may therefore only tolerate either of these two aliphatic residues. Thus if the isolate NK165 was to genetically drift in this position, the most likely amino acid substituted in place of original A (alanine) would be V (valine) given rise to novel variant 1. Likewise at Seq No2 HA position 23, only R (arginine) and K (lysine), both of which are basic residues, are found and tolerated at this position. If the isolate 1-KAN-1 is to genetically drift at this position, then the current K (lysine) would likely be replaced by R (arginine) giving rise to variant 2.

| | | |
|---|---|---|
| | 10 | 23 |
| NK165 | pksswssheAsVgvssacpyqGKssffr | (SEQ ID NO:20) |
| variant 1 | pksswssheMsVgvssa↓cpyqGKssffr | (SEQ ID NO:20) |
| 1-KAN-1 | pksswssheAsLgvssacpyqRKssffr | (SEQ ID NO:20) |
| variant 2 | pksswssheAsLgvssacpyqRRssffr | (SEQ ↓D NO:20) |
| variant 3 | pksswssheVsLgvssacpyqGRssffr | (SEQ ID NO:20) |
| variant 4 | pksswssheVsLgvssacpyqRKssffr | (SEQ ID NO:20) |
| variant 5 | pksswssheVsLgvssacpyqRRssffr | (SEQ ID NO:20) |
| variant 6 | pksswssheVsVgvssacpyqGRssffr | (SEQ ID NO:20) |
| variant 7 | pksswssheVsVgvssacpyqRKssffr | (SEQ ID NO:20) |
| variant 8 | pksswssheVsVgvssacpyqRRssffr | (SEQ ID NO:20) |
| variant 9 | pksswssheAsVgvssacpyqGRssffr | (SEQ ID NO:20) |
| variant 10 | pksswssheAsVgvssacpyqRKssffr | (SEQ ID NO:20) |
| variant 11 | pksswssheAsVgvssacpyqRRssffr | (SEQ ID NO:20) |

In this case we would synthesize all sixteen H5N1 Seq No2 HA variations (five parental wild type and eleven combinatorial permutations) for inclusion in the vaccine formulation, represented as a subset of SEQ ID NO: 20, as follows:

Pksswsshe(V/A)s(L/V)gvssacpyq(R/G)(R/K)ssffr (SEQ ID NO:20)

### Example 1b

There may be instances where the retrieved human isolates would generate a much higher degree of combinatorial diversity.

A/Indonesia/286H/2006

(SEQ ID NO:21)

KIVLLLAIVSLVKSDQICIGYHANNSTEQVDTIMEKNVTVTHAQDILEKT
HNGKLCDLDGVKPLILRDCSVAGWLLGNPMCDEFINVPEWSYIVEKANPT
NDLCYPGSFNDYEELKHLLSRINHFEKIQIIPKSSWSDHEASSGVSSACP
YLGSPSFFRNVVWLIKKNSTYPTIKKSYNNTNQEDLLVLWGIHHPNDAAE
QTRLYQNPTTYISIGTSTLNQRLVPKIATRSKVNGQSGRMEFFWTILNPN
DAINFESNGNFIAPEYAYKIVKKGDSAIMKSELEYGNCNTKCQTPMGAIN
SSMPFHNIHPLTIGECPKYVKSNRLVLATGLRNSPQRESRRKKRGLFGAI
AGFIEGGWQGMVDGWYGYHHSNEQGSGYAADKESTQKAIDGVTNKVNSII
DKMNTQFEAVGREFNNLERRIENLNKKMEDGFLDVWTYNAELLVLMENER
TLDFHDSNVKNLYDKVRLQLRDNAKELGNGCFEFYHKCDNECMESIRNGT
YNYPQYSEEARLKREEISGVKLESIGTYQILSIYSTVASSLALAIMMAGL
SLWMCSNGSLQCRICI*ICEFRL*X

Building upon the example above, if we had included another more genetically distinctive isolate, A/Indonesia/286H (see above) the alignment of their respective Seq No2 HA portions would then be:

| | |
|---|---|
| 286H | |
| pksswsDheAsSgvssacpyLGSpsffr | (SEQ ID NO: 14) |
| NK165 | |
| pksswsSheAsVgvssacpyGGKssffr | (SEQ ID NO: 14) |
| 2-SP-33 | |
| pksswsSheVsLgvssacpyGGKssffr | (SEQ ID NO: 14) |
| Chaiyaphum/622 | |
| pksswsSheAsLgvssacpyGGRssffr | (SEQ ID NO: 14) |
| SP83: | |
| pksswsSheAsLgvssacpyGGKssffr | (SEQ ID NO: 14) |
| 1-KAN-1: | |
| pksswsSheAsLgvssacpyGRKssffr | (SEQ ID NO: 14) |

In this case after aligning Seq No2 HA, there are six variable positions (bold uppercase) when combined, would generate 144 different permutations. To limit the number of combinatorial variants for synthesis, we would choose only those variants that differ by one amino acid from any of the original wild-type parents.

For example, the 286H parental sequence and a resulting combinatorial variant 1 that differs in one position is shown below. Also shown is variant X that has incorporated three additional mutational shifts. We would not include variant X for future synthesis, as the four incorporated mutations are too drastic of a change and not likely to occur in the immediate evolution of the parental 286H.

| | |
|---|---|
| 286H WT | |
| pksswsDheAsSgvssacpyLGSssffr | (SEQ ID NO: 14) |
| 286H variant 1 | |
| pksswsDheAsLgvssacp↓yLRSssffr | (SEQ ID NO: 14) |
| 286H variant X | |
| pksswsSheAsLgvssacpyGRKssffr | (SEQ ID NO: 14) |

Further analysis enumerate thirty one combinatorial sequences (variants 1-31 below) that differ by one amino acid from one of the six wild-type parental sequences and comprise SEQ ID No14.

| | |
|---|---|
| variant 1 | |
| pksswsDheAsLgvssacpyLRSssffr | (SEQ ID NO: 14) |
| variant 2 | |
| pksswsDheAsVgvssacpyLGSpsffr | (SEQ ID NO: 14) |
| variant 3 | |
| pksswsDheAsVgvssacpyQGKssffr | (SEQ ID NO: 14) |
| variant 4 | |
| pksswsDheAsLgvssacpyQGrssffr | (SEQ ID NO: 14) |
| variant 5 | |
| pksswsDheAsLgvssacpyQGKssffr | (SEQ ID NO: 14) |
| variant 6 | |
| pksswsSheVsLgvssacpyQGRssffr | (SEQ ID NO: 14) |
| variant 7 | |
| pksswsSheAsLgvssacpyQGKpsffr | (SEQ ID NO: 14) |
| variant 8 | |
| pksswsSheAsVgvssacpyQGRssffr | (SEQ ID NO: 14) |
| variant 9 | |
| pksswsSheAsVgvssacpyQGKpsftr | (SEQ ID NO: 14) |
| variant 10 | |
| pksswsDheVsLgvssacpyQGKssffr | (SEQ ID NO: 14) |
| variant 11 | |
| pksswsDheAsSgvssacpyLGRpsffr | (SEQ ID NO: 14) |
| variant 12 | |
| pksswsDheVsSgvssacpyLGSpsffr | (SEQ ID NO: 14) |
| variant 13 | |
| pksswsSheAsVgvssacpyQGSssffr | (SEQ ID NO: 14) |
| variant 14 | |
| pksswsSheAsVgvssacpyLGKssffr | (SEQ ID NO: 14) |
| variant 15 | |
| pksswsSheAsSgvssacpyQGKssffr | (SEQ ID NO: 14) |
| variant 16 | |
| pksswsSheAsSgvssacpyQGRssffr | (SEQ ID NO: 14) |
| variant 17 | |
| pksswsSheAsSgvssacpyLGSpsffr | (SEQ ID NO: 14) |
| variant 18 | |
| pksswsSheVsLgvssacpyQGKpsffr | (SEQ ID NO: 14) |
| variant 19 | |
| pksswsSheAsLgvssacpyQGRpsffr | (SEQ ID NO: 14) |
| variant 20 | |
| pksswsSheVsLgvssacpyQGSssffr | (SEQ ID NO: 14) |
| variant 21 | |
| pksswsSheVsLgvssacpyLGKssffr | (SEQ ID NO: 14) |
| variant 22 | |
| pksswsSheLsLgvssacpyQGKssffr | (SEQ ID NO: 14) |
| variant 23 | |
| pksswsSheAsLgvssacpyQGSssffr | (SEQ ID NO: 14) |
| variant 24 | |
| pksswsSheVsSgvssacpyQGKssffr | (SEQ ID NO: 14) |
| variant 25 | |
| pksswsSheAsLgvssacpyLGKssffr | (SEQ ID NO: 14) |
| variant 26 | |
| pksswsDheAsLgvssacpyLGSpsffr | (SEQ ID NO: 14) |
| variant 27 | |
| pksswsSheAsLgvssacpyLGRssffr | (SEQ ID NO: 14) |
| variant 28 | |
| pksswsDheAsSgvssacpyLGSssffr | (SEQ ID NO: 14) |
| variant 29 | |
| pksswsDheAsSgvssacpyLGRpsffr | (SEQ ID NO: 14) |
| variant 30 | |
| pksswsDheAsSgvssacpyLGKpsffr | (SEQ ID NO: 14) |
| variant 31 | |
| pksswsDheAsSgvssacpyQGSpsffr | (SEQ ID NO: 14) |

### Example 2

### Eukaryotic Expression

A number of types of cells may act as suitable host cells for expression of the desired polypeptide(s). Eukaryotic and preferably mammalian host cells include: COS (monkey kidney cells) NIH 3T3 cells, Chinese hamster ovary (CHO) cells, HeLa cells, human kidney 293 cells, human epidermal A431 cells, and other cell lines, for in vitro culture. It is also possible to attain high yields of protein through the Sf9 baculovirus insect expression system.

Depending on the culture system chosen, differential glycosylation and other post-translational processing may occur between cell lines. These glycosylation differences can be initially determined and visualized by SDS-PAGE after immunoprecipitation by anti-HA antibodies. Differential glycosylation processing will be demonstrated by bands of unequal electrophoretic. A more precise method of determining the identity of the added N-glycan is through mass spectrometry. Mass spectrometry (MS) has been a powerful tool for the characterization of glycosylation including fast atom bombardment, matrix-assisted laser desorption ionization, and electrospray ionization (ESI) using a wide variety of mass analyzers. In tandem a second spectrophotometer bombards a selected ion with neutral atoms that cause further fragmentation. The resultant mass spectrometric spectrum can then provide sequence and oligosaccharide information.

Mass Spectrometry

In this example, influenza HA proteins can be prepared by the above cell culture system. The HA proteins can be purified by SDS-PAGE and recovered by membrane transfer for MALDI mass spectrometry. For tryptic peptide analysis, the samples are desalted with C₁₈ and eluted in 70% acetonitrile and 0.1% TFA. The samples are directly eluted onto 0.5 μL spots of -cyano-4-hydroxycinnamic acid (—CHCA) on the plate. After the solubilization of protein, the samples are analyzed immediately. Sinapinic acid matrix (50 mM 3,5-dimethoxy-4-hydroxy-cinnamic acid) in 70% formic acid are spotted onto the sample plate(s). Internal calibration standards are also included. A MALDI mass spectrometer (Voyager-DE STR Biospectrometry Workstation; Applied Biosystems, Foster City, Calif.) is applied using Angiotensin I, ACTH and bovine insulin as standards. For MALDI-TOF/TOF analysis, of peptides isolated from the in-gel digestion, these samples can be desalted with C₁₈ pipette tips (Zip Tips; Millipore) and mixed with —CHCA (1:1) before spotting onto the sample plate. A proteomics analyzer (model 4700; Applied Biosystems) is then used in positive ion mode with a laser intensity between 4200 and 4500 nm. Precursor ions are selected with a window of 10 and 1000 to 5000 shots were averaged for each spectrum.

Once the full mass spectrum (MS) is determined, the complex mass fingerprints can be analyzed by dedicated software programs. Given that the HA sequences are known, peptides can be identified using fragmentation information from MS spectra. The software can correlate theoretical MS data from a database with the actual data for identification.

Glycosylation Prediction

The analysis of derived combinatorial polypeptide sequences includes those which may be glycosylated at particular antigenic sites. Glycosylation sites can be predicted and then verified later using the above mass spectrophotometer analysis. There are a variety of prediction tools available. As stated above, the sequence motif Asn-Xaa-Ser/Thr (Xaa is any amino acid except Pro) has been defined as a prerequisite for N-glycosylation and many predictive search algorithms exploit these sites. Although rare, the sequence motif Asn-Xaa-Cys has also been shown to act as a N-glycosylation acceptor site.

Unlike N-glycosylation, there is no acceptor motif defined for O-linked glycosylation. The only common characteristic among most O-glycosylation sites is that they occur on serine and threonine residues in close proximity to proline residues, and that the acceptor site is usually in a beta-conformation. Both O-glycosylation and N-glycosylation pattern recognition employ some weight matrix algorithms in conjunction with amino acid positional sequence of in vivo data.

For O-glycosylation predictions, we have utilized the NetOglyc neural network predictor of mucin type O-glycosylation sites in mammalian proteins (J. E. Hansen, et al. Prediction of O-glycosylation of mammalian proteins: Specificity patterns of UDP-GalNAc:polypeptide N-acetylgalactosaminyltransferase. The Biochemical Journal, 308, 801-813, 1995.) For N-glycosylation predictions, we have utilized the NetNglyc neural network predictor server based on (R. Gupta, et al., Center for Biological Sequence analysis, Technical University of Denmark).

### Example 3

Mice

BALB/c mice (6-12 weeks old) are purchased from the Jackson Laboratory and are maintained in a specific pathogen-free isolation environment.

Immunization

Mice are immunized with multiple (four) doses of the vaccine candidate peptides as described above. Synthetic peptides can be synthesized while cell culture expression candidates, either free or as fusion proteins, are purified by successive chromatography. Approximately 50 μg per mouse for each immunization can be dosed over a three week interval on days 0, 7, 14, and 21 in combination with different adjuvant formulations using various sites of administration: intrarectal (IR), intranasally or subcutaneously. For subcutaneous immunization, incomplete Freund's adjuvant was used. Control animals receive carrier only.

Antigen Specific Antibody Titer Analysis

Blood collection and NA or HA-specific antibody endpoint titer are collected before and after immunization. Specific antibody determination is performed by serial dilution of the sera before application to 96-well enzyme-linked immunosorbent assay (ELISA) plates. The wells are plated with either detergent disrupted influenza virus or coated with the vaccine candidate peptides and then blocked with (1% bovine serum albumin (BSA) in PBS. Blood samples are then added for approximately two hours before washing the plates for non-specific binding. After washing with 0.05% tween-20/PBS, the wells are treated with Horse Radish Peroxidase (HRP)-labeled anti-mouse IgG antibody. HRP substrate (3,3′-diaminobenzidine tetrahydrochloride dihydrate in 50 mM Tris.HCl, pH 7.5, containing 0.015% hydrogen peroxide) is then applied and OD values determined to calculate specific antibody dilution ranges.

In Vitro Assays for T-Cell Responses

Peripheral blood mononuclear cells (PBMCs) from study animals are harvested and then separated from the coagulated blood using Ficoll-Hypaque density gradient separation. The interface includes mononuclear cells which are then washed and grown in culture media (RPMI, 10% fetal calf serum and added specific cytokines such as IL-2). Test vaccine peptide, 5-500 □M are then typically first pulsed onto adherent antigen presenting cells supplemented with exogenous beta-2-microglobulin. Donor lymphocytes can be specifically enriched for CD8+ (CTL) or CD4+ (Thelper) cells, before or after peptide stimulation, using positive selection with anti-CD8 or anti-CD4 columns or magnetic beads, or passing cells over columns of antibody-coated nylon-coated steel wool or FACS sorting. Isolated CD8+ and/or CD4+ Lymphocytes are restimulated usually once or twice a week with autologous PBMCs or cells that have been irradiated and pulsed with the stimulated peptide. After several rounds of stimulation, and when a significant number of peptide-specific cells have been generated, in vitro assays of T-cell responses can be initiated. These can cytoxicity assays, proliferation assays, cytokine assays, FACS analyses, limiting dilution, ELISPOT.

Cytotoxicity Assay

Activated CD8+ T cells generally kill any cells that display the specific peptide:MHC complex they recognize. Target APC cells are radiolabeled with ⁵¹Cr or ³⁵M and plated together with peptide-specific T-cells at various effector:target ratios. Typical ratios are 100:1, 50:1, 25:1, and 12.5:1. Cells are incubated together for 4-16 hours and culture medium is collected for measurement of radioactive label that has been released from lysed cells. Radiolabeled cells incubated for the same period of time without T-cell cultures give represent background release of radioactive label.

Proliferation Assay (³HTdR Incorporation into DNA)

Target cells are irradiated and incubated together with peptide-specific T-cells at various effector:target ratios. At certain time points, ³H thymidine is added to the culture and after overnight growth and DNA incorporation, cells are lysed and the radioactivity is measured as an indication of the amount of proliferation of the T-cell population.

Cytokine Release Assays

One method to measure the responses of T-cell populations is a variant of the antigen-capture ELISA method, called the ELISPOT assay. In this assay, cytokine secreted by individual activated T cells is immobilized as discrete spots on a plastic plate via anti-cytokine antibodies, which are counted to give the number of activated T cells. Ninety-six-well nitrocellulose plates (Milititer HA, Millipore) are coated with 7 μg/ml of a monoclonal antibody against mouse IFN-gamma (PharMingen) in 75 μl PBS and incubated at room temperature overnight. Wells are washed six times with culture medium and incubated with DMEM supplemented with 10% FCS for 1 h at 37° C. Pooled splenocytes from immunized mice were added to antibody-coated wells in serial dilutions. P815 cells (a mastocytoma cell line that expresses only MHC class 1 molecules) were used as antigen-presenting cells. P815 cells (1×105 cells/ml) were pulsed with 1×10⁻⁶ M of the synthetic vaccine candidate peptide (see Seq IDs above) for 1 h at 37° C. After repeated washings with culture medium, cells were treated with 50 μg/ml mitomycin C (Sigma) for 1 h. NP-peptide treated P815 cells were added to each well. As a control, untreated P815 cells were used. Plates were incubated for 24 h at 37° C. with 5% CO₂ and then washed extensively with PBS+0.05% Tween-20 (PBS/T). Wells are then incubated with a solution of 2 μg/ml biotinylated anti-mouse IFN-gamma monoclonal antibody (PharMingen) in PBS/T for 1 h at room temperature. Plates are washed with PBS/T and incubated with peroxidase-labeled streptavidin for 1 h at room temperature. Wells were washed with PBS/T and PBS, and 1 μg/ml substrate (3,3′-diaminobenzidine tetrahydrochloride dihydrate in 50 mM Tris.HCl, pH 7.5, containing 0.015% hydrogen peroxide) was added. Spots in each well are then counted with the aid of a microscope.

Another method is to collect culture supernatant from stimulated cells and measure cytokines directly by standard ELISA methods. To test the cytokine profile produced by individual cells, intracellular cytokine staining relies on the use of metabolic poisons to inhibit protein export from the cell. The cytokine thus accumulates within the endoplasmic reticulum and vesicular network of the cells. Once cells are fixed and permeabilized, antibodies can gain access to the intracellular compartments to detect cytokine, using flow cytometry.

Flow Cytometry

The activation state of in vitro peptide-stimulated T-cells can be assessed using fluorescence-activated cell sorter or FACS. Cells are washed free of culture medium and incubated with isotype control or specific anti-CD antibody for 1 hr. at 4° C. Either the first antibody or a secondary antibody is labeled with a fluorescent marker. After washing cells free of unbound antibody, they are collected and analyzed by a FACS machine. The percentage of positive cells or the intensity of the fluorescence can give an indication of the activation state of the cells. For examples, markers of T-cell activation include CD69 and CD25, the IL-2 receptor alpha chain. In addition, flow cytometry can be used to detect fluorescently labeled cytokines within activated T cells or the directly detect T cells on the basis of the specificity of their receptor, using fluorochrome-tagged tetramers of specific MHC:peptide complexes.

Influenza Virus Challenge

Influenza A viruses were grown in embryonated chicken eggs between the ages of 6 and 14 days old (SPAFAS, Preston, Conn.) at 37° C. for 48 h. Influenza B viruses were grown in embryonated chicken eggs at 35° C. for 72 h. One hundred plaque-forming units (pfu) of virus were injected into the allantoic cavity of each egg. Allantoic fluid from influenza A or B virus-infected eggs was serially diluted in PBS and assayed for hemagglutination (HA) of chicken red blood cells (0.5%; Truslow Farms, Chestertown, Md.) in 96-well plates. Plaque assay of influenza A or B virus stocks was performed on Madin-Darby canine kidney cells (MDCK) cells in the presence of 2 μg/ml trypsin (Difco) at 37° C. (influenza A viruses) or 35° C. (influenza B viruses).

BALB/c mice are an established model for influenza viral infection (Neirynck, S., et al. (1999) Nat. Med. 5, 1157-1163). Under light diethyl ether anesthesia, mice were infected simultaneously by the intratracheal route with five lethal doses (LD50) of influenza A (strain of choice) or in PBS using 24-gauge stainless steel feeding animal needle (All animal work is conducted under BL3 conditions). The infected and control mice are observed for a period from 0 to 28 days, and resultant mortality rates calculated. For viral lung titers, mice were killed at either day 3 or day 6. Lungs were homogenized and resuspended in sterile PBS (100 mg lung tissue per 1 ml PBS) and titered on MDCK cells in the presence of 2 μg/ml trypsin.

Serologic Tests

If the mice above are able to produce anti-influenza specific antibodies, the protective nature of these antibodies can be assayed using MDCK neutralization assays. Neutralization assays were done by mixing 100 ID50 of virus (strain of choice) and test antisera for 1 h at 23° C.; this is followed by titration of the mixtures for residual virus infectivity on MDCK cell monolayers in 96-well plates. After 3 days of incubation at 37° C. in 5% CO2, neutralization titers were assessed for the presence of a cytopathic effect in the cultures and for HA activity in the supernatant. Neutralization titers are then expressed as the reciprocal of the antibody dilution that completely inhibited virus infectivity in 50% of triplicate cultures.

Immunoprecipitation of Influenza from MDCK Infected Cells

Dishes of 70% confluent MDCK cells were infected [multiplicity of infection (MOI)=10] with wild-type influenza virus, or mock infected with PBS for 1 h at room temperature. Cells are then incubated in 1 ml labeling media (MEM-cys/met+250 μCi 35S-cys/met) for 8 h at 37° C. and lysed on ice in 200 μl RIPA buffer (0.15 mM NaCl/0.05 mM Tris.HCl, pH 7.2/1% Triton X-100/1% sodium deoxycholate/0.1% SDS). Lysates are clarified by centrifugation for 10 min at 12,000×g. Cell supernatant was precipitated for 2 h at 4° C. by using a polyclonal antibody (2 μl) against the influenza A virus. GeneTex, Inc. San Antonio, Tex. Fifty microliters of Protein A Sepharose beads is added to mixture and antibody complexes are pelleted after incubation at 4° C. for 1 h. Pellets were washed twice with RIPA buffer, and half of the samples were loaded onto 17.5% SDS/PAGE gel. Separated bands were visualized by autoradiography.

Influenza Infection and Monitoring of Ferrets

Young adult male or female ferrets (Marshall Farms, North Rose, N.Y.) aged 8 to 10 months and serologically negative by hemagglutination inhibition assay for test strain influenza A or B viruses are moved at least 4 days prior to infection to BSL-3 animal holding area, and housed in cages contained in bioclean portable laminar flow clean room enclosures (Lab Products, Seaford, Del.). Prior to infection, their baseline temperature is measured twice daily for at least 3 days. Ferrets are then anesthetized with ketamine (25 mg/kg), xylazine (2 mg/kg), and atropine (0.05 mg/kg) by the intramuscular route and infected intranasally (i.n.) with a total of 1 ml of 10⁷ EID50 of virus/ml in PBS delivered to the nostrils. Control animals are mock infected with an equivalent dilution (1:30) of noninfectious allantoic fluid used to prepare the virus. Temperatures are measured twice daily using either a rectal thermometer or a subcutaneous implantable temperature transponder (BioMedic Data Systems, Inc., Seaford, Del.). Pre-infection values were averaged to obtain a baseline temperature for each ferret. The change in temperature (in degrees Celsius) is calculated at each time point for each animal. Clinical signs of sneezing (before anesthesia), inappetence, dyspnea, and level of activity are also assessed daily. A scoring system based on that described by (Reuman et al. 1989. Assessment of signs of influenza illness in the ferret model. J. Virol. Methods 24:27-34.)

can be used to assess the activity level. Based on the daily scores for each animal in a group, a relative inactivity index was calculated as follows: % (day 1 to day 7) [score ! 1]n/% (day 1 to day 7) n, where n equals the total number of observations. A value of 1 was added to each base score so that a score of 0 could be divided by a denominator, resulting in an index value of 1.0. The numbers of animals assessed on different days were as follows: days 0 and 1, n & 9; day 3, n & 7; day 5, n & 5; and days 7 and 9, n & 3.

The FID50 was determined for each virus by i.n. infection of two ferrets each with doses of 10⁴, 10³, and 10² EID50 of virus and three ferrets each with 10¹ EID50 of virus as described above. Nasal wash samples were collected on day 3 p.i. and titrated in eggs to detect the infectious virus. Animals with nasal wash titers of 10² EID50/ml were considered positive for virus.

It is claimed:

1. An Influenza vaccine composition comprising, in a physiological carrier,
a subset of 5-50 peptide antigens having amino acid sequences contained in the set of peptides identified by a sequences selected from the group consisting of SEQ ID NOS: 1-13, where each of SEQ ID NOS. 1-13 represents a selected region from one of the major influenza surface antigens, hemagglutinin (HA) and neuraminidase (NA), as follows:
Seq No 1 HA: (residues 211 to 240; including the 190-helix (residues 223-231); Seq No 2 HA: (residues 151 to 180; including the 130-loop (residues 165-168); Seq No 3 HA: (residues 151 to 180; including the 220-loop (residues 254-261); Seq No 4 HA: (residues 366 to 394; including the “Cleavage site” {residue 380} where for full infectivity, the single chain (HA0) is cut into two chains for full infectivity; and Seq No 5: NA (residues 18 to 437), Seq No 6: NA (residues 321 to 341), Seq No 7: NA (residues 342 to 400), Seq No 8: (residues 153 to 185), Seq No 9: (residues 209 to 232), Seq No 10: (residues 330 to 369), Seq No 11: (residues 369 to 398), Seq No 12: (residues 399 to 434), and Seq No 13: (residues 435 to 460).
said subset of peptide antigens being selected from the total set of antigen peptides defined by one of SEQ ID NOS: 1-13 by the steps of:
(i) limiting the influenza-strain isolates examined for amino acid variations within one of the regions defining SEQ ID NOS: 1-13 to a selected one of the human-infective influenza subtypes identified by H1, H2, H3, and H5;
(ii) optionally, limiting the influenza-strain variants examined for amino-acid variation to those associated with a particular geographic region;
(iii) selecting for the subset, peptide antigens having amino acid sequences that represent existing amino acid variants examined in steps (i) and (ii), and those that represent most-likely amino acid mutations of one or more of the existing variants, such that the total number of peptide antigens selected for the subset is between 5 and 50.

2. The influenza vaccine composition of claim 1, wherein the subset of peptide antigens is selected from the total set of combinatorial antigen peptides defined by SEQ ID NO: 2 by the steps of:
step (i) includes limiting the influenza-strain variants examined for amino acid variations within SEQ ID NO: 2 to H5N1 subtypes of the virus;
step (ii) includes limiting the influenza-strain variants examined for amino-acid variation to those associated with human influenza infections in Indonesia and Thailand, and
step (iii) includes selecting for the subset, 6 peptide antigens having amino acid sequences that represent existing amino acid variants examined in steps (i) and (ii), and 31 single-amino acid mutations of one or more of the existing variants, such that the total number of peptide antigens selected for the subset is 37 and the sequence of the subset is given by SEQ ID NO:14.

3. The influenza vaccine composition of claim 1, wherein the subset of peptide antigens is selected from the total set of combinatorial antigen peptides defined by SEQ ID NO: 2 by the steps of:
step (i) includes limiting the influenza-strain variants examined for amino acid variations within SEQ ID NO: 2 to H5N1 subtypes of the virus;
step (ii) includes limiting the influenza-strain variants examined for amino-acid variation to those associated with human influenza infections in Indonesia and Thailand, and
step (iii) includes selecting for the subset, 5 peptide antigens having amino acid sequences that represent existing amino acid variants examined in steps (i) and (ii), and 11 single-amino acid mutations of one or more of the existing variants, such that the total number of peptide antigens selected for the subset is 16 and the sequence of the subset is given by SEQ ID NO:20.

4. A method of producing an influenza vaccine composition comprising, in a physiological carrier, a subset of 5-50 peptide antigens having amino acid sequences substantially contained in the set of peptides identified by a sequences selected from the group consisting of SEQ ID NOS: 1-13, where each of SEQ ID NOS. 1-13 represents a selected region from one of the major influenza surface antigens, hemagglutinin (HA) and neuraminidase (NA), as follows:
Seq No 1 HA: (residues 211 to 240; including the 190-helix (residues 223-231); Seq No 2 HA: (residues 151 to 180; including the 130-loop (residues 165-168); Seq No 3 HA: (residues 151 to 180; including the 220-loop (residues 254-261); Seq No 4 HA: (residues 366 to 394; including the “Cleavage site” {residue 380} where for full infectivity, the single chain (HA0) is cut into two chains for full infectivity; and Seq No 5: NA (residues 18 to 437), Seq No 6: NA (residues 321 to 341), Seq No 7: NA (residues 342 to 400), Seq No 8: (residues 153 to 185), Seq No 9: (residues 209 to 232), Seq No 10: (residues 330 to 369), Seq No 11: (residues 369 to 398), Seq No 12: (residues 399 to 434), and Seq No 13: (residues 435 to 460), said method comprising the steps of:
(i) limiting the influenza-strain variants examined for amino acid variations within one of the regions defining SEQ ID NOS: 1-13 to a selected one of the human-infective influenza subtypes identified by H1, H2, H3, H5; H7, and H9,
(ii) optionally, limiting the influenza-strain variants examined for amino-acid variation to those associated with a particular geographic region; and
(iii) selecting for the subset, peptide antigens having amino acid sequences that represent existing amino acid variants examined in steps (i) and (ii), and those that represent most-likely amino acid mutations of one or more of the existing variants, such that the total number of peptide antigens selected for the subset is between 5 and 50.


SEQUENCE LISTING (21 sequences)

SEQ ID NO: 1 - protein, 29 aa, Artificial Sequence
  Synthetic Peptide Antigen
XXWGXHHPXXXAEQXXLYXNXXXXXXXXX

SEQ ID NO: 2 - protein, 29 aa, Artificial Sequence
  Synthetic Peptide Antigen
PXXSXWXXXXXXXXXXGXXXACXXXXFXR

SEQ ID NO: 3 - protein, 30 aa, Artificial Sequence
  Synthetic Peptide Antigen
XXXXXXXPXIXXXRPXVXGXXXRXXXWXXX

SEQ ID NO: 4 - protein, 22 aa, Artificial Sequence
  Synthetic Peptide Antigen
XPXXXXXGLFGAIAGFIEGXGW

SEQ ID NO: 5 - protein, 42 aa, Artificial Sequence
  Synthetic Peptide Antigen
ATLAVYCNSLXIIKXPXDXXENGXEXXXVYSXKPINDARQTX

SEQ ID NO: 6 - protein, 41 aa, Artificial Sequence
  Synthetic Peptide Antigen
LVGDTPRKNDXXSSSHCLXPNNEEGGHGVKGWAFDDXNDVW

SEQ ID NO: 7 - protein, 39 aa, Artificial Sequence
  Synthetic Peptide Antigen
MGRTIXEKXRXGYETFKVIEGWSXPNSKLQINRQVIVXR

SEQ ID NO: 8 - protein, 33 aa, Artificial Sequence
  Synthetic Peptide Antigen
XQGXXLXXKHSNXTVXDXXPXRTLXXXXAGXXX

SEQ ID NO: 9 - protein, 24 aa, Artificial Sequence
  Synthetic Peptide Antigen
LXXXIXGXDNXAXAXXXYXGXXXD

SEQ ID NO: 10 - protein, 36 aa, Artificial Sequence
  Synthetic Peptide Antigen
XXXXXYXCSGXXGDXPXPNDGXXXCXXXXXNXXXGV

SEQ ID NO: 11 - protein, 31 aa, Artificial Sequence
  Synthetic Peptide Antigen
KGXXFXXGNXYXWXGRTXSXXXRSGXEXXXX

SEQ ID NO: 12 - protein, 37 aa, Artificial Sequence
  Synthetic Peptide Antigen
XXGWXXXXSSXXXXQXIIXEXXDXXGYSGXFXXQHPX

SEQ ID NO: 13 - protein, 25 aa, Artificial Sequence
  Synthetic Peptide Antigen
XXGXXCXXXCFXVELIRGRXXEXXX

SEQ ID NO: 14 - protein, 28 aa, Artificial Sequence
  Synthetic Peptide Antigen
PKSSWSXHEXSXGVSSACPYXXXSSFFR

SEQ ID NO: 15 - protein, 571 aa, Artificial Sequence
  Synthetic Peptide Antigen
RIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKPNPANDLCYPGDFNDYEELKHLLSRINHFEKIQIIPKSSWSSHEASVGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQREKRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIKLESD

SEQ ID NO: 16 - protein, 572 aa, Artificial Sequence
  Synthetic Peptide Antigen
KIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEVSLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAAAKESTQKAIDGVTNKVNSIIDKMNTQFEAVGREFNNLERRIENLNKKMEDGFLDVWTYNAELLVLMENERTLDFHDSNVKNLYDKVRLQLKDNAKELGNGCFEFYHKCDNECMESVRNGTYDYPQYSEEARLKREEISGVKLESIGIYQILSIYSTVASSLALAIMVAGLSLWMCSNGSLQCRICIICEFRL

SEQ ID NO: 17 - protein, 573 aa, Artificial Sequence
  Synthetic Peptide Antigen
KIVLLFAMVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGRSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEIGGVKLESIGIYQILSIYSTVASSLALAIMVAGLSLWMCSNGSLQCRICIICEFRLL

SEQ ID NO: 18 - protein, 573 aa, Artificial Sequence
  Synthetic Peptide Antigen
KIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIICEFRLL

SEQ ID NO: 19 - protein, 574 aa, Artificial Sequence
  Synthetic Peptide Antigen
KIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPKDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGTTHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIKICESRLR

SEQ ID NO: 20 - protein, 28 aa, Artificial Sequence
  Synthetic Peptide Antigen
PKSSWSSHEXSXGVSSACPYQXXSSFFR

SEQ ID NO: 21 - protein, 572 aa, Artificial Sequence
  Synthetic Peptide Antigen
KIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILNPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICIICEFRL